(19) United States
(12) Patent Application Publication
(10) Pub. No.: US 2010/0022517 A1
(43) Pub. Date: Jan. 28, 2010

(54) OPHTHALMIC FORMULATION OF RHO KINASE INHIBITOR COMPOUND

(76) Inventors: Lori A. Richards, Wake Forest, NC (US); Christopher S. Crean, Pittsboro, NC (US); Ward M. Peterson, Morrisville, NC (US); Leo A. Trevino, Hurdle Mills, NC (US)

Correspondence Address: HOWERY LLP, C/O IP DOCKETING DEPARTMENT, 2941 FAIRVIEW PARK DRIVE SUITE 200, FALLS CHURCH, VA, 22042, US

(21) Appl. No.: 12/483,175

(22) Filed: Jun. 11, 2009

Related U.S. Application Data
(63) Continuation-in-part of application No. 11/958,214, filed on Dec. 17, 2007.
(60) Provisional application No. 60/870,555, filed on Dec. 18, 2006.
(60) Provisional application No. 61/073,519, filed on Jun. 18, 2008.

(51) Int. Cl. A61K 31/55; A61K 31/454; A61P 27/02

(52) U.S. Cl. 514/217.09; 514/322

(57) ABSTRACT

The present invention relates to an aqueous pharmaceutical formulation comprising at least one inhibitor of Rho-associated protein kinase (ROCK). The aqueous pharmaceutical formulation comprises 0.01-0.4% w/v of ROCK inhibitor(s), a non-ionic surfactant in an amount of 0.01-2% w/v, and a tonicity agent to maintain a tonicity between 220-360 mOsm/kG, at a pH between 6.3 to 7.8, wherein the ROCK inhibitor, the surfactant, and the tonicity agent are compatible in the formulation. The aqueous ophthalmic formulations of this invention have an increased ocular bioavailability and/or aqueous humor concentrations without a concomitant increase in systemic concentrations. The present invention further provides a method of reducing intraocular pressure, particularly a method of treating glaucoma, by administering the aqueous pharmaceutical formulation to a subject.

This application is a continuation-in-part of U.S. patent application Ser. No. 11/958,214, filed Dec. 17, 2007; which claims the benefit of U.S. Provisional Application 60/870,555, filed Dec. 18, 2006. This application also claims the benefit of U.S. Provisional Application No. 61/073,519, filed Jun. 18, 2008. The contents of the above-identified applications are incorporated herein by reference in their entirety.

### TECHNICAL FIELD

This invention relates to pharmaceutical formulations, particularly aqueous ophthalmic formulations, of Rho Kinase (ROCK) inhibitor compounds and their related analogs. The invention also relates to using such formulations for treating diseases or disorders by altering the integrity or rearrangement of the cytoskeleton, particularly, for treating disorders in which intraocular pressure (IOP) is elevated, such as primary open-angle glaucoma.

### BACKGROUND OF THE INVENTION

The Rho family of small GTP binding proteins can be activated by several extracellular stimuli such as growth factors, hormones and mechanic stress and function as a molecular signaling switch by cycling between an inactive GDP-bound form and an active GTP-bound form to elicit cellular responses. Rho-kinase (ROCK) functions as a key downstream mediator of Rho and exists as two isoforms (ROCK 1 and ROCK 2) that are ubiquitously expressed. ROCKs are serine/threonine kinases that regulate the function of a number of substrates including cytoskeletal proteins such as adducing, moesin, Na⁺—H⁺ exchanger 1 (NHE1), LIM-kinase and vimentin, contractile proteins such as the myosin light chain phosphatase binding subunit (MYPT-1), CPI-17, myosin light chain and calponin, microtubule associated proteins such as Tau and MAP-2, neuronal growth cone associate proteins such as CRMP-2, signaling proteins such as PTEN and transcription factors such as serum response factor (Loirand et al, Circ Res 98:322-334 (2006)). ROCK is also required for cellular transformation induced by RhoA. As a key intermediary of multiple signaling pathways, ROCK regulates a diverse array of cellular phenomena including cytoskeletal rearrangement, actin stress fiber formation, proliferation, chemotaxis, cytokinesis, cytokine and chemokine secretion, endothelial or epithelial cell junction integrity, apoptosis, transcriptional activation and smooth muscle contraction. As a result of these cellular actions, ROCK regulates physiologic processes such as vasoconstriction, bronchoconstriction, tissue remodeling, inflammation, edema, platelet aggregation and proliferative disorders.

One well documented example of ROCK activity is in smooth muscle contraction, In smooth muscle cells ROCK mediates calcium sensitization and smooth muscle contraction. Agonists (noradrenaline, acetylcholine, endothelin, etc.) that bind to G protein coupled receptors produce contraction by increasing both the cytosolic Ca²⁺ concentration and the Ca²⁺ sensitivity of the contractile apparatus. The Ca²⁺-sensitizing effect of smooth muscle constricting agents is ascribed to ROCK-mediated phosphorylation of MYPT-1, the regulatory subunit of myosin light chain phosphatase (ML CP), which inhibits the activity of MLCP resulting in enhanced phosphorylation of the myosin light chain and smooth muscle contraction (WO 2005/003101A2, WO 2005/034866A2).

Glaucoma is an ophthalmic disease that leads to irreversible visual impairment. It is the fourth most common cause of blindness and the second most common cause of visual loss in the United States, and the most common cause of irreversible visual loss among African-Americans. Generally speaking, the disease is characterized by a progressive optic neuropathy caused at least in part by deleterious effects resulting from increased intraocular pressure. In normal individuals, intraocular pressures ranges from 12 to 20 mm Hg, averaging approximately 16 mm Hg. However, in individuals suffering from primary open angle glaucoma, intraocular pressures generally rise above 22 to 30 mm Hg. In angle closure or acute glaucoma intraocular pressure can reach as high as 70 mm Hg leading to blindness within only a few days. Interestingly, the loss of vision can result from statistically normal intraocular pressures in individuals with unusually pressure-sensitive eyes; a condition known as normotensive glaucoma. [See, e.g., P. L. Kaufman and T. W. Mittag, “Medical Therapy Of Glaucoma,” Ch. 9, Sec. II (pp. 9.7-9.30) In P. L. Kaufman and T. W. Mittag (eds.): Glaucoma (Vol. 7 of S. M. Podos and M. Yanoff (eds): Textbook of Opthalmology Series). London, Mosby-Year Book Europe Ltd. (1994); A. C. Guyton, Textbook of Medical Physiology (W. B. Saunders Co., Sixth Ed.), pp. 386-89 (1981)].

Open-angle glaucoma constitutes approximately 90% of all primary glaucomas and is characterized by abnormally high resistance to fluid (aqueous humor) drainage from the eye. Normal resistance is required to maintain an intraocular pressure sufficient to maintain the shape of the eye for optical integrity. This resistance is provided by the trabecular meshwork, a complex, multilaminar tissue consisting of specialized cells with a dense actomyosin cytoskeleton network, collagenous beams and extracellular matrix. The resistance of the trabecular meshwork normally is such that intraocular pressure is 16 mm Hg, a pressure at which aqueous humor leaves the eye at the same rate at which it is produced (2.5 μL/minute). In the glaucomatous eye, the rate of aqueous humor production remains constant, while it is the increased resistance to outflow that is responsible for the elevated intraocular pressure.

Typical treatments for glaucoma comprise a variety of pharmaceutical approaches for reducing intraocular pressure (IOP), each with their drawbacks. Beta-blockers and carbonic anhydrase inhibitors reduce aqueous humor production, which is needed to nourish the avascular lens and corneal endothelial cells, and the prostaglandins effect the uvealscleral outflow pathway, which only accounts for 10% of the total outflow facility. There are currently no commercially approved therapeutic agents which act directly upon the trabecular meshwork, the site of aqueous humor drainage where increased resistance to aqueous humor outflow is responsible for elevated IOP. Therefore, a medical need remains for improved IOP-lowering medications that target this structure. Pharmacological agents which target the trabecular meshwork may provide relief to the significant numbers of patients that do not respond adequately to current IOP-lowering medications and/or cannot tolerate the side effects associated with these agents. Additionally, these molecules may prove beneficial as adjunctive therapy in combination with other classes of IOP-lowering medications.

U.S. Pat. Nos. 6,586,425, 6,110,912, and 5,798,380 disclose a method for the treatment of glaucoma using compounds that affect the actin filament integrity of the eye to enhance aqueous humor outflow. These patents specifically disclose latrunculin-A, -latrunculin-B, swinholide-A, and jasplakinolide, which cause a perturbation of the actin cytoskeleton and tight junctional complexes in the trabecular meshwork or the modulation of its interactions with the underlying membrane.

U.S. Pat. Nos. 6,649,625 and 6,673,812 disclose the pharmaceutical use of certain compounds having a Rho kinase inhibitory activity for the treatment of glaucoma.

It has long been recognized that systemic absorption of topically applied glaucoma drugs via the conjuctival and nasal mucosae during ocular drug therapy may elicit significant side effects [eg., Nelson, W. L., Fraundfelder, F. T., Sills, J. M., Arrowsmith J. B., Kuritsky, J. N., “Adverse respiratory cardiac events attributed to timolol ophthalmic solution,” Am. J. Opthalmol. 1986, 102, pp 606-11; Saxena, R.; Prakash, J., Mathur, P., Gupta, S. K. “Pharmacotherapy of Glaucoma,” Ind. J. Pharmacol, 2002, 34, pp 71-85].

There is a need for improved IOP-lowering drug formulations that have increased drug bioavailability but without a concomitant increase of the drug concentration in systemic circulation, which would therefore reduce or eliminate the unwanted drug-associated side effects.

### SUMMARY OF THE INVENTION

The present invention is directed to an aqueous pharmaceutical formulation comprising at least one ROCK inhibitor of Formula II in an amount of 0.01-0.4% w/v, a non-ionic surfactant in an amount of 0.01-2% w/v, and a tonicity agent to maintain a tonicity between 220-360 mOsm/kG, at a pH between 6.3 to 7.8, wherein the ROCK inhibitor, the surfactant, and the tonicity agent are compatible in the formulation.

A preferred surfactant of the formulation is a polysorbate, a polaxamer, or a combination thereof. Preferred pH of the formulation is 6.3-7.5. More preferred pH of the formulation is 6.3-7.3. The formulation optionally comprises a chelating agent and/or a preservative. The tonicity agent can be non-ionic such as glycerol, mannitol, or dextrose. The tonicity agent can also be ionic such as sodium chloride.

The aqueous ophthalmic formulations of this invention have an increased ocular bioavailability and/or aqueous humor concentrations without a concomitant increase in systemic concentrations.

The present invention further provides a method of reducing intraocular pressure, particularly a method of treating glaucoma, by identifying a subject in need of treatement and administering the aqueous pharmaceutical formulation to the subject.

### BRIEF DESCRIPTION OF THE FIGURES

FIG. 1 shows the stability of Compound A at pH 5.3, 6.3 and 7.3 at 60° C. After the formulations were stored at 60° C. for a period of time, percent of Compound A remaining were determined and shown in FIG. 1.

FIG. 2A shows the aqueous humor (AH) Cmax after a 0.12% dose of Compound A at pH 5.3, 6.3 and 7.3. FIG. 2B shows the aqueous humor AUC after a 0.12% dose of Compound A at pH 5.3, 6.3 and 7.3. Data represents mean±SEM, N=4 eyes.

FIG. 3A shows the Plasma Cmax after a 0.12% dose of Compound A at pH 5.3, 6.3 and 7.3. FIG. 3B shows the plasma AUC after a 0.12% dose of Compound A at pH 5.3, 6.3 and 7.3. Data represents mean±SEM, N=2 animals.

FIG. 4 shows the ocular surface concentration over time after a 0.12% (3 mM) dose of Compound A at pH 5.3, 6.3 and 7.3, Data represents mean±SEM, N=4 eyes.

FIG. 5 shows ocular comfort scores of different formulations.

FIG. 6 shows ocular comfort scores of different concentrations of Compound 2.039, in comparison with other drugs.

### DETAILED DESCRIPTION OF THE INVENTION

### Definitions

When present, unless otherwise specified, the following terms are generally defined as, but are not limited to, the following:

Halo substituents are taken from fluorine, chlorine, bromine, and iodine.

“Alkyl” refers to groups of from 1 to 12 carbon atoms inclusively, either straight chained or branched, more preferably from 1 to 8 carbon atoms inclusively, and most preferably 1 to 6 carbon atoms inclusively.

“Alkenyl” refers to groups of from 2 to 12 carbon atoms inclusively, either straight or branched containing at least one double bond but optionally containing more than one double bond.

“Alkynyl” refers to groups of from 2 to 12 carbon atoms inclusively, either straight or branched containing at least one triple bond but optionally containing more than one triple bond, and additionally optionally containing one or more double bonded moieties.

“Alkoxy” refers to the group alkyl-O— wherein the alkyl group is as defined above including optionally substituted alkyl groups as also defined above.

“Alkenoxy” refers to the group alkenyl-O— wherein the alkenyl group is as defined above including optionally substituted alkenyl groups as also defined above.

“Alkynoxy” refers to the group alkynyl-O— wherein the alkynyl group is as defined above including optionally substituted alkynyl groups as also defined above.

“Aryl” refers to an unsaturated aromatic carbocyclic group of from 6 to 14 carbon atoms inclusively having a single ring (e.g., phenyl) or multiple condensed rings (e.g., naphthyl or anthryl). Preferred aryls include phenyl, naphthyl and the like.

“Arylalkyl” refers to aryl-alkyl-groups preferably having from 1 to 6 carbon atoms inclusively in the alkyl moiety and from 6 to 10 carbon atoms inclusively in the aryl moiety. Such arylalkyl groups are exemplified by benzyl, phenethyl and the like.

“Arylalkenyl” refers to aryl-alkenyl-groups preferably having from 2 to 6 carbon atoms in the alkenyl moiety and from 6 to 10 carbon atoms inclusively in the aryl moiety.

“Arylalkynyl” refers to aryl-alkynyl-groups preferably having from 2 to 6 carbon atoms inclusively in the alkynyl moiety and from 6 to 10 carbon atoms inclusively in the aryl moiety.

“Cycloalkyl” refers to cyclic alkyl groups of from 3 to 12 carbon atoms inclusively having a single cyclic ring or multiple condensed rings which can be optionally substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple ring structures such as adamantyl, and the like.

“Cycloalkenyl” refers to cyclic alkenyl groups of from 4 to 12 carbon atoms inclusively having a single cyclic ring or multiple condensed rings and at least one point of internal unsaturation, which can be optionally substituted with from 1 to 3 alkyl groups. Examples of suitable cycloalkenyl groups include, for instance, cyclobut-2-enyl, cyclopent-3-enyl, cyclooct-3-enyl and the like.

“Cycloalkylalkyl” refers to cycloalkyl-alkyl-groups preferably having from 1 to 6 carbon atoms inclusively in the alkyl moiety and from 6 to 10 carbon atoms inclusively in the cycloalkyl moiety. Such cycloalkylalkyl groups are exemplified by cyclopropylmethyl, cyclohexylethyl and the like.

“Cycloalkylalkenyl” refers to cycloalkyl-alkenyl-groups preferably having from 2 to 6 carbon atoms inclusively in the alkenyl moiety and from 6 to 10 carbon atoms inclusively in the cycloalkyl moiety. Such cycloalkylalkenyl groups are exemplified by cyclohexylethenyl and the like.

“Cycloalkylalkynyl” refers to cycloalkyl-alkynyl-groups preferably having from 2 to 6 carbon atoms inclusively in the alkynyl moiety and from 6 to 10 carbon atoms inclusively in the cycloalkyl moiety. Such cycloalkylalkynyl groups are exemplfied by cyclopropylethynyl and the like.

“Heteroaryl” refers to a monovalent aromatic heterocyclic group of from 1 to 10 carbon atoms inclusively and 1 to 4 heteroatoms inclusively selected from oxygen, nitrogen and sulfur within the ring, Such heteroaryl groups can have a single ring (e.g., pyridyl or furyl) or multiple condensed rings (e.g., indolizinyl or benzothienyl).

“Heteroarylalkyl” refers to heteroaryl-alkyl-groups preferably having from 1 to 6 carbon atoms inclusively in the alkyl moiety and from 6 to 10 atoms inclusively in the heteroaryl moiety. Such heteroarylalkyl groups are exemplified by pyridylmethyl and the like.

“Heteroarylalkenyl” refers to heteroaryl-alkenyl-groups preferably having from 2 to 6 carbon atoms inclusively in the alkenyl moiety and from 6 to 10 atoms inclusively in the heteroaryl moiety.

“Heteroarylalkynyl” refers to heteroaryl-alkynyl-groups preferably having from 2 to 6 carbon atoms inclusively in the alkynyl moiety and from 6 to 10 atoms inclusively in the heteroaryl moiety.

“Heterocycle” refers to a saturated or unsaturated group having a single ring or multiple condensed rings, from 1 to 8 carbon atoms inclusively and from 1 to 4 hetero atoms inclusively selected from nitrogen, sulfur or oxygen within the ring. Such heterocyclic groups can have a single ring (e.g., piperidinyl or tetrahydrofuryl) or multiple condensed rings (e.g., indolinyl, dihydrobenzofuran or quinuclidinyl). Preferred heterocycles include piperidinyl, pyrrolidinyl and tetrahydrofuryl.

“Heterocycle-alkyl” refers to heterocycle-alkyl-groups preferably having from 1 to 6 carbon atoms inclusively in the alkyl moiety and from 6 to 10 atoms inclusively in the heterocycle moiety. Such heterocycle-alkyl groups are exemplified by morpholino-ethyl, pyrrolidinylmethyl, and the like.

“Heterocycle-alkenyl” refers to heterocycle-alkenyl-groups preferably having from 2 to 6 carbon atoms inclusively in the alkenyl moiety and from 6 to 10 atoms inclusively in the heterocycle moiety.

“Heterocycle-alkynyl” refers to heterocycle-alkynyl-groups preferably having from 2 to 6 carbon atoms inclusively in the alkynyl moiety and from 6 to 10 atoms inclusively in the heterocycle moiety.

Examples of heterocycles and heteroaryls include, but are not limited to, furan, thiophene, thiazole, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, pyrrolidine, indoline and the like.

Unless otherwise specified, positions occupied by hydrogen in the foregoing groups can be further substituted with substituents exemplified by, but not limited to, hydroxy, oxo, nitro, methoxy, ethoxy, alkoxy, substituted alkoxy, trifluoromethoxy, haloalkoxy, fluoro, chloro, bromo, iodo, halo, methyl, ethyl, propyl, butyl, alkyl, alkenyl, alkynyl, substituted alkyl, trifluoromethyl, haloalkyl, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, phenyl, aryl, substituted aryl, aryloxy, arylalkyl, arylalkenyl, arylalkynyl pyridyl, imidazolyl, heteroaryl, substituted heteroaryl, heteroaryloxy, heteroarylalkyl, heteroarylalkenyl, heteroarylalkynyl, cyclopropyl, cyclobutyl, cyclopentyl, cyclohexyl, cycloalkyl, cycloalkenyl, cycloalkylalkyl, substituted cycloalkyl, cycloalkyloxy, pyrrolidinyl, piperidinyl, morpholino, heterocycle, (heterocycle)oxy, and (heterocycle)alkyl; and preferred heteroatoms are oxygen, nitrogen, and sulfur. It is understood that where open valences exist on these substituents they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further understood that the above subtitutions can be made provided that replacing the hydrogen with the substituent does not introduce unacceptable instability to the molecules of the present invention, and is otherwise chemically reasonable.

The term “heteroatom-containing substituent” refers to substituents containing at least one non-halogen heteroatom. Examples of such substituents include, but are not limited to, hydroxy, oxo, nitro, methoxy, ethoxy, alkoxy, substituted alkoxy, trifluoromethoxy, haloalkoxy, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, aryloxy, pyridyl, imidazolyl, heteroaryl, substituted heteroaryl, heteroaryloxy, heteroarylalkyl, heteroarylalkenyl, heteroarylalkynyl, cycloalkyloxy, pyrrolidinyl, piperidinyl, morpholino, heterocycle, (heterocycle)oxy, and (heterocycle)alkyl; and preferred heteroatoms are oxygen, nitrogen, and sulfur. It is understood that where open valences exist on these substituents they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further understood that the above subtitutions can be made provided that replacing the hydrogen with the substituent does not introduce unacceptable instability to the molecules of the present invention, and is otherwise chemically reasonable.

“Pharmaceutically acceptable salts” are salts that retain the desired biological activity of the parent compound and do not impart undesired toxicological effects. Pharmaceutically acceptable salt forms include various polymorphs as well as the amorphous form of the different salts derived from acid or base additions. The acid addition salts can be formed with inorganic or organic acids. Illustrative but not restrictive examples of such acids include hydrochloric, hydrobromic, sulfuric, phosphoric, citric, acetic, propionic, benzoic, napthoic, oxalic, succinic, gentisic, maleic, fumaric, malic, adipic, lactic, tartaric, salicylic, methanesulfonic, 2-hydroxyethanesulfonic, toluenesulfonic, benzenesulfonic, camphorsulfonic, and ethanesulfonic acids. The pharmaceutically acceptable base addition salts can be formed with metal or organic counterions and include, but are not limited to, alkali metal salts such as sodium or potassium; alkaline earth metal salts such as magnesium or calcium; and ammonium or tetraalkyl ammonium salts, i.e., NX₄⁺ (wherein X is C₁₋₄).

“Tautomers” are compounds that can exist in one or more forms, called tautomeric forms, which can interconvert by way of a migration of one or more hydrogen atoms in the compound accompanied by a rearrangement in the position of adjacent double bonds. These tautomeric forms are in equilibrium with each other, and the position of this equilibrium will depend on the exact nature of the physical state of the compound. It is understood that where tautomeric forms are possible, the current invention relates to all possible tautomeric forms.

“Solvates” are addition complexes in which a compound of Formula I or Formula II is combined with a pharmaceutically acceptable cosolvent in some fixed proportion. Cosolvents include, but are not limited to, water, methanol, ethanol, 1-propanol, isopropanol, 1-butanol, isobutanol, tert-butanol, acetone, methyl ethyl ketone, acetonitrile, ethyl acetate, benzene, toulene, xylene(s), ethylene glycol, dichloromethane, 1,2-dichloroethane, N-methylformamide, N,N-dimethylformamide, N-methylacetamide, pyridine, dioxane, and diethyl ether. Hydrates are solvates in which the cosolvent is water. It is to be understood that the definitions of compounds in Formula I and Formula II encompass all possible hydrates and solvates, in any proportion, which possess the stated activity.

This invention is directed to a pharmaceutical formulation comprising ROCK inhibitor compounds and their related analogs.

There are five key constraints to develop a successful topical formulation for treating glaucoma: solubility, formulation stability, ocular surface tolerability, systemic exposure, and aqueous humor exposure. Ocular surface relates to the surface of the cornea and conjunctiva. Ocular surface residence time is the average time that a drug resides on the ocular surface. Aqueous humor is the fluid within the anterior chamber of the eye and has the closest correlation to the concentration at the site of action, the trabecular meshwork. Rarely do the physicochemical properties of any drug allow for all five constraints to be simultaneously optimal under one condition. The ideal ophthalmic formulation for treating glaucoma is: (1) sufficiently soluble to deliver a therapeutic dose of drug; (2) sufficiently stable to have a commercial product shelf-life as a glaucoma medication; (3) well-tolerated on the ocular surface at the therapeutic dose; and (4) achieves therapeutic level of drug in the aqueous humor (the site of action) with minimal systemic exposure, thereby minimizing systemic side effects.

For ROCK inhibitor compounds of the present invention, the inventors have discovered that pH of the formulation can influence three of the five constraints. Lower pH improves the solubility and stability of ROCK inhibitor compounds in an aqueous formulation. However, the inventors have discovered that higher pH of the formulation increases residence time of the compound on the ocular surface, thus allowing more drugs to penetrate into the anterior chamber and resulting in higher compound concentration in aqueous humor. The inventors have discovered that pH of the formulation (from pH 5.3-7.3) does not have an effect on systemic exposure. The inventors do not find evidence that pH of the formulation affects ocular comfort up to pH 7.3. However, the ocular surface is in general more tolerable in an acidic formulation than in a basic formulation.

The inventors have identified and selected a pH range of pH 6.3-7.8 that allows for acceptable sacrifices or trade-offs between solubility, stability, ocular tolerability, systemic exposure, and aqueous humor exposure (for example, poorer solubility may be acceptable at a certain pH if greater tolerability or exposure is achieved).

The concentrations of the ROCK inhibitor compounds affect the ocular comfort; i.e., lower concentrations are more comfortable. The inventors have identified and selected a concentration range of 0.01-0.4% (w/v) of ROCK inhibitor compounds in the formulation; such concentration is effective to provide a therapeutic effect and does not cause ocular discomfort.

The inventors have unexpectedly discovered that certain ROCK-inhibiting compounds, whose aqueous solubilities decrease with increasing pH, exhibit significant increase in anterior chamber bioavailability as the formulation pH is adjusted from acidic pH to physiologic pH over a pharmaceutically acceptable pH range for topical ophthalmic formulations (pH 4.5 to pH 7.8). The inventors have also unexpectedly discovered that ROCK-inhibiting compounds of the present invention increase residence time on the ocular surface and increase concentrations in aqueous humor when the pH of the ophthalmic formulations increases. The increase in residence time on the ocular surface and anterior chamber bioavailability allows lower concentrations of drugs to be used to treat diseases and disorders associated with cytoskeleton disruption, such as primary open-angle glaucoma.

The inventors have discovered that systemic exposure, as measured by the plasma concentration, is only related to the ROCK inhibitor concentration and volume of the ophthalmic formulation, and is not related to pH or composition of the formulation. Therefore, by providing an ophthalmic formulation with a pH of 6.3-7.8, which is closer to a neutral pH but is higher than that of commonly used ophthalmic formulations, the formulation increases the anterior chamber bioavailability through an increase in membrane penetration, but does not concomitantly increase the systemic exposure; thereby increasing the potential ocular to systemic therapeutic margin.

Thus, despite the limitations that this pH of 6.3-7.8 poses for solubility and stability, the exposure in the aqueous humor is sufficiently increased to counterbalance the limitations. The present invention provides optimal therapeutic benefit-risk without having to expose the ocular surface with a high concentration of drug.

### Rho Kinase Inhibitor Compounds

The Rho kinase inhibitor compounds useful for this invention include compounds of general Formula I and Formula II, and/or tautomers thereof, and/or pharmaceutically-acceptable salts, and/or solvates, and/or hydrates thereof.

A compound according to Formula I or Formula II can exist in several diastereomeric forms. The general structures of Formula I and Formula II include all diastereomeric forms of such materials, when not specified otherwise. Formula I and Formula II also include mixtures of compounds of these Formulae, including mixtures of enantiomers, diastereomers and/or other isomers in any proportion.

### A. Formula I

Compounds of Formula I are as follows:

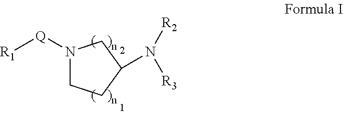

wherein: R₁ is aryl or heteroaryl, optionally substituted;
Q is C═O, SO₂, or (CR₄R₅)ₙ₃;
n₁ is 1, 2, or 3;
n₂ is 1 or 2;
n₃ is 0, 1, 2, or 3;
wherein the ring represented by

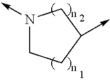

is optionally substituted by alkyl, halo, oxo, OR₆, NR₆R₇, or SR₆;
R₂ is selected from the following heteroaryl systems, optionally substituted:

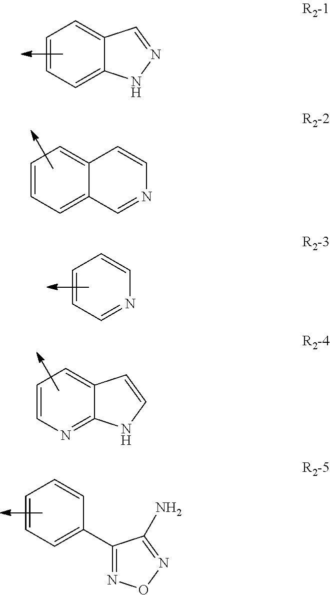

R₃-R₇ are independently H, alkyl, alkenyl, alkynyl, cycloalkyl, cycloalkenyl, cycloalkylalkyl, cycloalkylalkenyl, or cycloalkylalkynyl optionally substituted. In Formula I, the preferred R₁ is substituted aryl, the more preferred R₁ is substituted phenyl, the preferred Q is (CR₄R₅)ₙ₃, the more preferred Q is CH₂, the preferred n₁ is 1 or 2, the preferred n₂ is 1, the preferred n₃ is 1 or 2, and the preferred R₃-R₇ are H.
[1] One embodiment of the invention is represented by Formula I, in which R₂ is 5-indazolyl or 6-indazolyl (R₂-1), optionally substituted.
[1a] In embodiment 1, R₂-1 is substituted by one or more alkyl or halo substituents.
[1b] In embodiment 1, R₂-1 is substituted by one or more amino, alkylamino, hydroxyl, or alkoxy substituents.
[1c] In embodiment 1, R₂-1 is unsubstituted.
[2] In another embodiment, the invention is represented by Formula I in which R₂ is 5-isoquinolinyl or 6-isoquinolinyl (R₂-2), optionally substituted.
[2a] In embodiment 2, R₂-2 is substituted by one or more alkyl or halo substituents.
[2b] In embodiment 2, R₂-2 is substituted by one or more amino, alkylamino, hydroxyl, or alkoxy substituents.
[2c] In embodiment 2, R₂-2 is unsubstituted.
[3] In another embodiment, the invention is represented by Formula I in which R₂ is 4-pyridyl or 3-pyridyl (R₂-3), optionally substituted.
[3a] In embodiment 3, R₂-3 is substituted by one or more alkyl or halo substituents.
[3b] In embodiment 3, R₂-3 is substituted by one or more amino, alkylamino, hydroxyl, or alkoxy substituents.
[3c] In embodiment 3, R₂-3 is unsubstituted.
[4] In another embodiment, the invention is represented by Formula I in which R₂ is 7-azaindol-4-yl or 7-azaindol-5-yl (R₂-4), optionally substituted.
[4a] In embodiment 4, R₂-4 is substituted by one or more alkyl or halo substituents.
[4b] In embodiment 4, R₂-4 is substituted by one or more amino, alkylamino, hydroxyl, or alkoxy substituents.
[4c] In embodiment 4, R₂-4 is unsubstituted,
[5] In another embodiment, the invention is represented by Formula I in which R₂ is 4-(3-amino-1,2,5-oxadiazol-4-yl)phenyl or 3-(3-amino-1,2,5-oxadiazol-4-yl)phenyl (R₂-5), optionally substituted.
[5a] In embodiment 5, R₂-5 is unsubstituted.
[6] In another embodiment, the invention is represented by Formula I in which R₂ is one of the groups R₂-1-R₂-5, substituted by one or more alkyl, halo, amino, alkylamino, hydroxyl, or alkoxy substituents,
[6a] In embodiment 6, R₂ is substituted by one or more alkyl or halo substituents.
[6b] In embodiment 6, R₂ is substituted by one or more amino, alkylamino, hydroxyl, or alkoxy substituents.
[7] In another embodiment, the invention is represented by Formula I in which R₂ is one of the groups R₂-1-R₂-5, and is unsubstituted.
[8] In another embodiment, the invention is represented by Formula I in which R₃ is H.
[9] In another embodiment, the invention is represented by Formula I in which Q is (CR₄R₅)ₙ₃, and n₃ is 1 or 2.
[10] In another embodiment, the invention is represented by Formula I in which Q is (CH₂)ₙ₃, and n₃ is 1.
[11] In another embodiment, the invention is represented by Formula I in which R₁ is aryl or heteroaryl substituted with one or more alkenyl, alkynyl, aryl, arylalkyl, arylallenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituerts, optionally further substituted.

Compounds exemplifying embodiment 11 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table I.

[12] In another embodiment, the invention is represented by Formula I in which R₁ is aryl or heteroaryl substituted with one or more heteroatom-containing substituents, with the proviso that if the R₁ substituent is acyclic and is connected to R₁ by a carbon atom, then this substituent contains at least one nitrogen or sulfur atom, with the second proviso that if the substituent is acyclic and is connected to R₁ by an oxygen or nitrogen atom, then this substituent contains at least one additional oxygen, nitrogen or sulfur atom, and with the third proviso that if the substituent is connected to R₁ by a sulfone linkage “—SO₂—”, then R₂ is not nitrogen- or oxygen-substituted R₂-2.
[12a] In embodiment 12, the heteroatom-containing substituent is connected to R₁ by an oxygen or nitrogen atom.
[12b] In embodiment 12, the heteroatom-containing substituent is connected to R₁ by a sulfide linkage, “—S—”.
Compounds exemplifying embodiment 12 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table I.
[13] In another embodiment, the invention is represented by Formula I in which R₁ is aryl or heteroaryl substituted with one or more alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, which are further substituted with one or more heteroatom-containing substituents, with the proviso that if the R₁ substituent is acyclic and its heteroatom-containing substituent falls on the carbon by which it is attached to R₁, then the heteroatom-containing substituent contains at least one nitrogen or sulfur atom.
Compounds exemplifying embodiment 13 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, and 1.122, shown below in Table 1.
[14] In another embodiment, the invention is represented by Formula I in which R₁ is aryl or heteroaryl substituted with one or more alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, optionally further substituted, and R₂ is 5-indazolyl (R₂-1) or 5-isoquinolinyl (R₂-2), optionally substituted.
[14a] In embodiment 14, R₂ is 5-indazolyl (R₂-1), optionally substituted by one or more alkyl, halo, amino, alkylamino, hydroxyl, or alkoxy substituents.
[14b] In embodiment 14, R₂ is 5-isoquinolinyl (R₂-2), optionally substituted by one or more alkyl, halo, amino, alkylamino, hydroxyl, or alkoxy substituents.
[14c] In embodiment 14, R₂ is unsubstitued.
Compounds exemplifying embodiment 14 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table I.
[15] In another embodiment, the invention is represented by Formula I in which R₁ is aryl or heteroaryl substituted with one or more heteroatom-containing substituents, and R₂ is 5-indazolyl (R₂-1) or 5-isoquinolinyl (R₂-2), optionally substituted, with the proviso that if the R₁ substituent is acyclic and is connected to R₁ by a carbon atom, then this substituent contains at least one nitrogen or sulfur atom, with the second proviso that if the substituent is acyclic and is connected to R₁ by an oxygen or nitrogen atom, then this substituent contains at least one additional oxygen, nitrogen or sulfur atom, and with the third proviso that if the substituent is connected to R₁ by a sulfone linkage “—SO₂—”, then R₂ is not nitrogen- or oxygen-substituted R₂-2.
[15a] In embodiment 15, R₂ is 5-indazolyl (R₂-1), optionally substituted by one or more alkyl, halo, amino, alkylamino, hydroxyl, or alkoxy substituents.
   [15b] In embodiment 15, R₂ is 5-isoquinolinyl (R₂-2), optionally substituted by one or more alkyl, halo, amino, alkylamino, hydroxyl, or alkoxy substituents, [15c] In embodiment 15, R₂ is unsubstituted.
   [15d] In embodiment 15, the heteroatom-containing substituent is connected to R₁ by an oxygen or nitrogen atom.
   [15e] In embodiment 15, the heteroatom-containing substituent is connected to R₁ by a sulfide linkage, “—S-”.
   Compounds exemplifying embodiment 15 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table I.
   [16] In another embodiment, the invention is represented by Formula I in which R₁ is aryl or heteroaryl substituted with one or more alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, at least one of which is further substituted with one or more heteroatom-containing substituents, and R₂ is 5-indazolyl (R₂-1) or 5-isoquinolinyl (R₂-2), optionally substituted, with the proviso that if the R₁ substituent is acyclic and its heteroatom-containing substituent falls on the carbon by which it is attached to R₁, then the heteroatom-containing substituent contains at least one nitrogen or sulfur atom.
   [16a] In embodiment 16, R₂ is 5-indazolyl (R₂-1), optionally substituted by one or more alkyl, halo, amino, alkylamino, hydroxyl, or alkoxy substituents.
   [16b] In embodiment 16, R₂ is 5-isoquinolinyl (R₂-2), optionally substituted by one or more alkyl, halo, amino, alkylamino, hydroxyl, or alkoxy substituents.
   [16c] In embodiment 16, R₂ is unsubstituted.
   Compounds exemplifying embodiment 16 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, and 1.122, shown below in Table 1.

### B. Formula II

A preferred compound of Formula I is where R₁=Ar—X, shown below as Formula II:

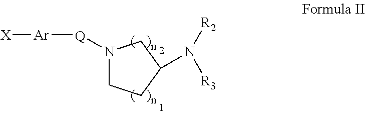

wherein:
Ar is a monocyclic or bicyclic aryl or heteroaryl ring, such as phenyl;
X is from 1 to 3 substituents on Ar, each independently in the form Y-Z, in which Z is attached to Ar;
Y is one or more substituents on Z, and each is chosen independently from H, halogen, or the heteroatom-containing substituents, including but not limited to OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀;
Each instance of Z is chosen independently from alkyl, alkenyl, alkynyl, aryl, arylalkyl, arylalkenyl, arylalkynyl, cycloalkyl cycloalkenyl, cycloalkylalkyl, cycloalkylalkenyl, cycloalkylalkynyl, heteroaryl, heteroarylalkyl, heteroarylalkenyl, heteroarylalkynyl, heterocycle, (heterocycle)alkyl, (heterocycle)alkenyl, (heterocycle)alkynyl, or is absent;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl; or heterocycle; optionally substituted by one or more halogen or heteroatom-containing substituents, including but not limited to OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, or NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents, including but not limited to OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, or NR₁₄C(═O)NR₁₅R₁₆;
any two of the groups R₈, R₉ and R₁₀ are optionally joined with a link selected from the group consisting of bond, —O—, —S—, —SO—, —SO₂—, and —NR₁₇— to form a ring;
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle.
In Formula II, the preferred Y is H, halogen, OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀, the more preferred Y is H, halogen, OR₈, SR₈, SOR₈, SO₂R₈, SO₂NR₈R₉, NR₈SO₂R₉, CONR₈R₉, or NR₈C(═O)NR₉R₁₀, the preferred Z is alkyl, alkenyl, alkynyl, cycloalkyl, cycloalkenyl, cycloalkylalkyl, or is absent; the more preferred Z is alkyl, alkenyl, alkynyl, cycloalkyl, or is absent, the preferred Q is (CR₄R₅)ₙ₃, the more preferred Q is CH₂, the preferred n₁ is 1 or 2, the preferred n2 is 1, the preferred n₃ is 1 or 2, the preferred R₃-R₇ are H, the preferred R₈ is H, alkyl, arylalkyl, cycloalkyl, cycloalkylalkyl, or heterocycle, the preferred R₈ substituents are H, halogen, OR₁₁, NR₁₁R₁₂, SR₁₁, SOR₁₁, SO₂R₁₁, SO₂NR₁₁R₁₂, NR₁₁SO₂R₁₂, CONR₁₁R₁₂, NR₁₁C(═O)R₁₂, and the preferred R₉-R₁₇ are H or alkyl.
[1] One embodiment of the invention is represented by Formula II in which R₂ is 5-indazolyl or 6-indazolyl (R₂-1), optionally substituted.
[1a] In embodiment 1, R₂-1 is substituted by one or more alkyl or halo substituents.
[1b] In embodiment 1, R₂-1 is substituted by one or more amino, alkylamino, hydroxyl, or alkoxy substituents.
[1c] In embodiment 1, R₂-1 is unsubstituted.
[2] In another embodiment, the invention is represented by Formula II in which R₂ is 5-isoquinolinyl or 6-isoquinolinyl (R₂-2), optionally substituted.
[2a] In embodiment 2, R₂-2 is substituted by one or more alkyl or halo substituents.
[2b] In embodiment 2, R₂-2 is substituted by one or more amino, alkylamino, hydroxyl, or alkoxy substituents.
[2c] In embodiment 2, R₂-2 is unsubstituted.
[3] In another embodiment, the invention is represented by Formula II in which R₂ is 4-pyridyl or 3-pyridyl (R₂-3), optionally substituted.
[3a] In embodiment 3, R₂-3 is substituted by one or more alkyl or halo substituents.
[3b] In embodiment 3, R₂-3 is substituted by one or more amino, alkylamino, hydroxyl, or alkoxy substituents.
[3c] In embodiment 3, R₂-3 is unsubstituted.
[4] In another embodiment, the invention is represented by Formula II in which R₂ is 7-azaindol-4-yl or 7-azaindol-5-yl (R₂-4), optionally substituted.
[4a] In embodiment 4, R₂-4 is substituted by one or more alkyl or halo substituents.
[4b] In embodiment 4, R₂-4 is substituted by one or more amino, alkylamino, hydroxyl, or alkoxy substituents.
[4c] In embodiment 4, R₂-4 is unsubstituted.
[5] In another embodiment, the invention is represented by Formula II in which R₂ is 4-(3-amino-1,2,5-oxadiazol-4-yl)phenyl or 3-(3-amino-1,2,5-oxadiazol-4-yl)phenyl (R₂-5), optionally substituted.
[5a] In embodiment 5, R₂-5 is unsubstituted.
[6] In another embodiment, the invention is represented by Formula II in which R₂ is one of the groups R₂-1-R₂-5, substituted by one or more alkyl, halo, amino, alkylamino, hydroxyl, or alkoxy substituents.
[6a] In embodiment 6, R₂ is substituted by one or more alkyl or halo substituents.
[6b] In embodiment 6, R₂ is substituted by one or more amino, alkylamino, hydroxyl, or alkoxy substituents.
[7] In another embodiment, the invention is represented by Formula II in which R₂ is one of the groups R₂-1-R₂-5, and is unsubstituted.
[8] In another embodiment, the invention is represented by Formula II in which R₃ is H.
[9] In another embodiment, the invention is represented by Formula II in which Q is (CR₄R₅)₃, and n₃ is 1 or 2.
[10] In another embodiment, the invention is represented by Formula II in which Q is (CH₂)ₙ₃, and n3 is 1.
[11] In another embodiment, the invention is represented by Formula II in which Z is alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkylalkenyl, cycloalkylalkynyl, cycloalkenyl, cycloalkylalkyl, heterocycle, (heterocycle)alkyl, (heterocycle)alkenyl, or (heterocycle)alkynyl.
Compounds exemplifying embodiment 11 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table I.
[12] In another embodiment, the invention is represented by Formula II in which Z is absent, Y is a heteroatom-containing substituent, including but not limited to OR₈, NR₈R₉, SR₈, SOR₈, SO₂R₈, SO₂NR₈R₉, NR₈SO₂R₉, CONR₈R₉, NR₈C(═O)R₉, NR₈C(═O)OR₉, OC(═O)NR₈R₉, or NR₈C(═O)NR₉R₁₀, with the proviso that if the substituent Y is acyclic and is connected to Ar by a carbon atom, then this substituent contains at least one nitrogen or sulfur atom, with the second proviso that if the substituent Y is acyclic and is connected to Ar by an oxygen or nitrogen atom, then this substituent contains at least one additional oxygen, nitrogen or sulfur atom, and with the third proviso that if the substituent Y is connected to Ar by a sulfone linkage “—SO₂-”, then R₂ is not nitrogen- or oxygen-substituted R₂-2.
[12a] In embodiment 12, the heteroatom-containing substituent is connected to R₁ by an oxygen or nitrogen atom.
[12b] In embodiment 12, the heteroatom-containing substituent is connected to R₁ by a sulfide linkage, “—S—”.
Compounds exemplifying embodiment 12 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table I.
[13] In another embodiment, the invention is represented by Formula II in which Z is alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl, and Y is a heteroatom-containing substituent, including but not limited to OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₁₀C(═O)OR₉, OC(═O)NR₈R₉, or NR₁₀C(═O)NR₉R₁₀, with the proviso that if Z is acyclic and Y falls on the carbon by which Z is attached to Ar, then Y contains at least one nitrogen or sulfur atom.
Compounds exemplifying embodiment 13 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, and 1.122, shown below in Table I.
[1,4] In another embodiment, the invention is represented by Formula II in which Z is alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl, and R₂ is 5-indazolyl (R₂-1) or 5-isoquinolinyl (R₂-2), optionally substituted.
[14a] In embodiment 14, R₂ is 5-indazolyl (R₂-1), optionally substituted by one or more alkyl, halo, amino, alkylamino, hydroxyl, or alkoxy substituents.
[14b] In embodiment 14, R₂ is 5-isoquinolinyl (R₂-2), optionally substituted by one or more alkyl, halo, amino, alkylamino, hydroxyl, or alkoxy substituents.
[14c] In embodiment 14, R₂ is unsubstituted. Compounds exemplifying embodiment 14 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table I.
[15] In another embodiment, the invention is represented by Formula II in which Z is absent, Y is a heteroatom-containing substituent, including but not limited to OR₈, NR₈R₉, SR₈, SOR₈, SO₂R₈, SO₂NR₈R₉, NR₈SO₂R₉, CONR₈R₉, NR₈C(═O)R₉, NR₈C(═O)OR₉, OC(═O)NR₈R₉, or NR₈C(═O)NR₉R₁₀, and R₂ is 5-indazolyl (R₂-1) or 5-isoquinolinyl (R₂-2), optionally substituted, with the proviso that if the substituent Y is acyclic and is connected to Ar by a carbon atom, then this substituent contains at least one nitrogen or sulfur atom, with the second proviso that if the substituent Y is acyclic and is connected to Ar by an oxygen or nitrogen atom, then this substituent contains at least one additional oxygen, nitrogen or sulfur atom, and with the third proviso that if the substituent Y is connected to Ar by a sulfone linkage “—SO₂-”, then R₂ is not nitrogen- or oxygen-substituted R₂-2.
[15a] In embodiment 15, R₂ is 5-indazolyl (R₂-1), optionally substituted by one or more alkyl, halo, amino, alkylamino, hydroxyl, or alkoxy substituents.
[15b] In embodiment 15, R₂ is 5-isoquinolinyl (R₂-2), optionally substituted by one or more alkyl, halo, amino, alkylamino, hydroxyl, or alkoxy substituents.
[15 c] In embodiment 15, R₂ is unsubstituted.
[15d] In embodiment 15, the heteroatom-containing substituent is connected to R₁ by an oxygen or nitrogen atom.
[15e] In embodiment 15, the heteroatom-containing substituent is connected to R₁ by a sulfide linkage, “—S—”.

Compounds exemplifying embodiment 15 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table I.

[1,6] In another embodiment, the invention is represented by Formula II in which Z is alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl, and Y is a heteroatom-containing substituent, including but not limited to OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀, and R₂ is 5-indazolyl (R₂-1) or 5-isoquinolinyl (R₂-2), optionally substituted, with the proviso that if Z is acyclic and Y falls on the carbon by which Z is attached to Ar, then Y contains at least one nitrogen or sulfur atom.
[16a] In embodiment 16, R₂ is 5-indazolyl (R₂-1), optionally substituted by one or more alkyl, halo, amino, alkylamino, hydroxyl, or alkoxy substituents.
[16b] In embodiment 16, R₂ is 5-isoquinolinyl (R₂-2), optionally substituted by one or more alkyl, halo, amino, alkylamino, hydroxyl, or alkoxy substituents.
[16c] In embodiment 16, R₂ is unsubstituted. Compounds exemplifying embodiment 16 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, and 1.122, shown below in Table 1.
In Embodiments 11-16 of Formula II, the preferred Q is (CR₄R₅)ₙ₃, the more preferred Q is CH₂, the preferred n₁ is 1 or 2, the preferred n₂ is 1, the preferred n₃ is 1 or 2, and the preferred R₃ is H.

The present compounds are useful for ophthalmic use, particularly in reducing intraocular pressure or treating glaucoma. To be therapeutically effective in ophthalmic use, the compounds must have both adequate potency and proper pharmacokinetic properties such as good permeability across the ocular surface. In general, compounds bearing polar functionality have preferred absorption properties and are particularly suitable for topical optical use. In general, compounds bearing small lipophilic functional groups have good ROCK inhibitory potency.

R₁ substitution in Formula I and X in Formula II are important factors for pharmacokinetic properties and ROCK inhibitory potency. Specifically, compounds bearing polar functionality, especially those specified in the embodiments 11, 12, 13, 14, 15, and 16 in Formulae I and II, above, are particularly suitable for topical optical use with adequate ROCK inhibiting activity. Compounds bearing small lipophilic functional groups, as specified in the embodiments 11, 12, 13, 14, 15, and 16 in Formulae I and II, above, display ROCK inhibition with adequate ocular permeability.

Specific compounds illustrative of Formula I and Formula II are shown in the following Table I. The example compounds have been numbered in such a way that numbers of the form 1.nnn indicate compounds in which R₂ is R₂-1, numbers of the form 2.nnn indicate compounds in which R₂ is R₂-2, and so on in a similar fashion for the remaining compound numbers and groups R₂. In the following structures, hydrogens are omitted from the drawings for the sake of simplicity. Tautomers drawn represent all tautomers possible. Structures are drawn to indicate the preferred stereochemistry; where stereoisomers may be generated in these compounds, structures are taken to mean any of the possible stereoisomers alone or a mixture of stereoisomers in any ratio.

TABLE I
Example Compounds.

| Compound | Structure | Embodiments |
|---|---|---|
| 1.001 | 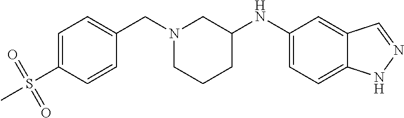 N-(1-(4-(methylsulfonyl)benzyl)piperidin-3-yl)-1H- indazol-5-amine | 1c, 7, 8, 9, 10, 12, 15c |
| 1.002 | 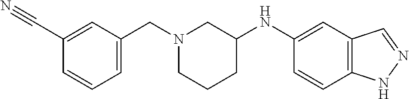 3-((3-(1H-indazol-5-ylamino)piperidin-1- yl)methyl)benzonitrile | 1c, 7, 8, 9, 10, 12, 15c |
| 1.003 | 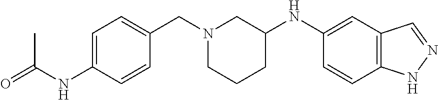 N-(4-((3-(1H-indazol-5-ylamino)piperidin-1- yl)methyl)phenyl)acetamide | 1c, 7, 8, 9, 10, 12a, 15c, 15d |
| 1.004 | 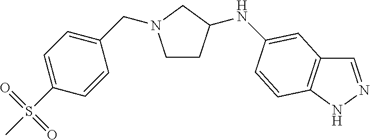 N-(1-(4-(methylsulfonyl)benzyl)pyrrolidin-3-yl)-1H- indazol-5-amine | 1c, 7, 8, 9, 10, 12, 15c |
| 1.005 | 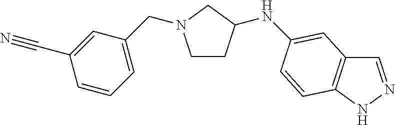 3-((3-(1H-indazol-5-ylamino)pyrrolidin-1- yl)methyl)benzonitrile | 1c, 7, 8, 9, 10, 12, 15c |
| 1.006 | 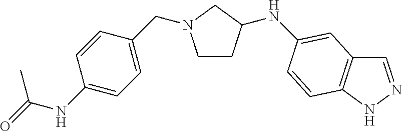 N-(4-((3-(1H-indazol-5-ylamino)pyrrolidin-1- yl)methyl)phenyl)acetamide | 1c, 7, 8, 9, 10, 12a, 15c, 15d |
| 1.007 | 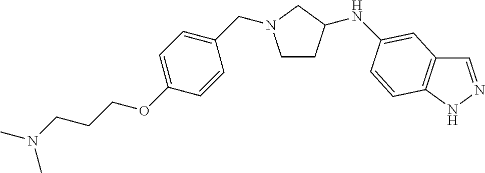 N-(1-(4-(3-(dimethylamino)propoxy)benzyl)pyrrolidin-3- yl)-1H-indazol-5-amine | 1c, 7, 8, 9, 10, 12a, 15c, 15d |
| 1.008 | 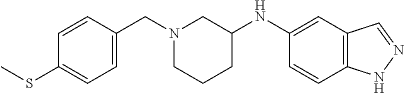 N-(1-(4-(methylthio)benzyl)piperidin-3-yl)-1H-indazol-5- amine | 1c, 7, 8, 9, 10, 12b, 15c, 15e |
| 1.009 | 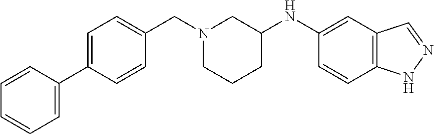 N-(1-(biphenyl-4-ylmethyl)piperidin-3-yl)-1H-indazol-5- amine | 1c, 7, 8, 9, 10, 11, 14c |
| 1.010 | 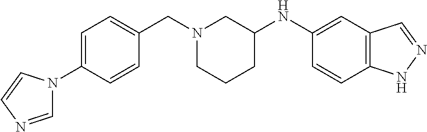 N-(1-(1H-imidazol-1-yl)benzyl)piperidin-3-yl)-1H- indazol-5-amine | 1c, 7, 8, 9, 10, 11, 14c |
| 1.011 | 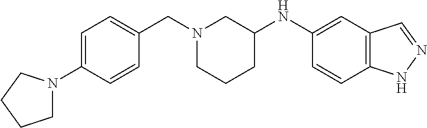 N-(1-(4-(pyrrolidin-1-yl)benzyl)piperidin-3-yl)-1H- indazol-5-amine | 1c, 7, 8, 9, 10, 11, 14c |
| 1.012 | 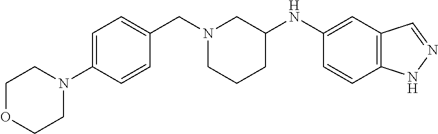 N-(1-(4-morpholinobenzyl)piperidin-3-yl)-1H-indazol-5- amine | 1c, 7, 8, 9, 10, 11, 14c |
| 1.013 | 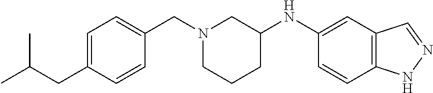 N-(1-(4-isobutylbenzyl)piperidin-3-yl)-1H-indazol-5- amine | 1c, 7, 8, 9, 10 |
| 1.014 | 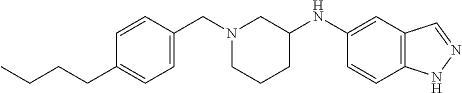 N-(1-(4-butylbenzyl)piperidin-3-yl)-1H-indazol-5-amine | 1c, 7, 8, 9, 10 |
| 1.015 | 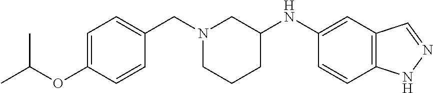 N-(1-(4-isopropoxybenzyl)piperidin-3-yl)-1H-indazol-5- amine | 1c, 7, 8, 9, 10 |
| 1.016 | 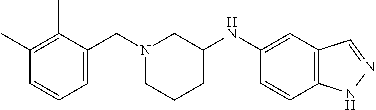 N-(1-(2,3-dimethylbenzyl)piperidin-3-yl)-1H-indazol-5- amine | 1c, 7, 8, 9, 10 |
| 1.017 | 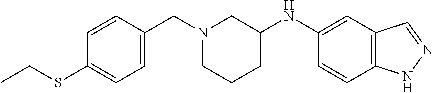 N-(1-(4-(ethylthio)benzyl)piperidin-3-yl)-1H-indazol-5- amine | 1c, 7, 8, 9, 10, 12b, 15c, 15e |
| 1.018 | 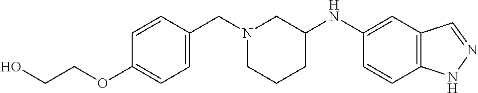 2-(4-((3-(1H-indazol-5-ylamino)piperidin-1-yl)methyl) phenoxy)ethanol | 1c, 7, 8, 9, 10, 12a, 15c, 15d |
| 1.019 | 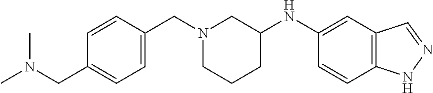 N-(1-(4-((dimethylamino)methyl)benzyl)piperidin-3-yl)- 1H-indazol-5-amine | 1c, 7, 8, 9, 10, 13, 16c |
| 1.020 | 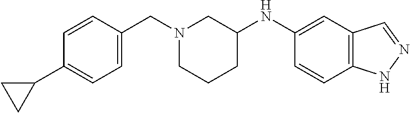 N-(1-(4-cyclopropylbenzyl)piperidin-3-yl)-1H-indazol-5- amine | 1c, 7, 8, 9, 10, 11, 14c |
| 1.021 | 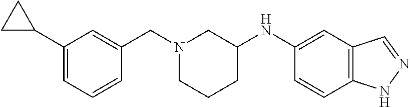 N-(1-(3-cyclopropylbenzyl)piperidin-3-yl)-1H-indazol-5- amine | 1c, 7, 8, 9, 10, 11, 14c |
| 1.022 | 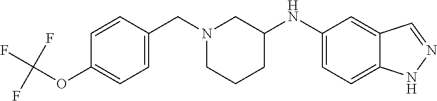 N-(1-(4-(trifluoromethoxy)benzyl)piperidin-3-yl)-1H- indazol-5-amine | 1c, 7, 8, 9, 10 |
| 1.023 | 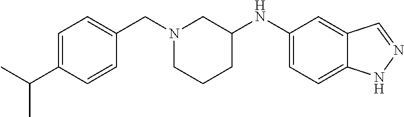 N-(1-(4-isopropylbenzyl)piperidin-3-yl)-1H-indazol-5- amine | 1c, 7, 8, 9, 10 |
| 1.024 | 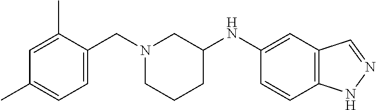 N-(1-(2,4-dimethylbenzyl)piperidin-3-yl)-1H-indazol-5- amine | 1c, 7, 8, 9, 10 |
| 1.025 | 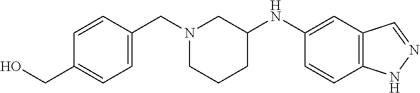 (4-((3-(1H-indazol-5-ylamino)piperidin-1- yl)methyl)phenyl)methanol | 1c, 7, 8, 9, 10 |
| 1.026 | 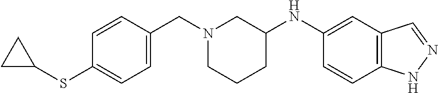 N-(1-(4-(cyclopropylthio)benzyl)piperidin-3-yl)-1H- indazol-5-amine | 1c, 7, 8, 9, 10, 12b, 15c, 15e |
| 1.027 | 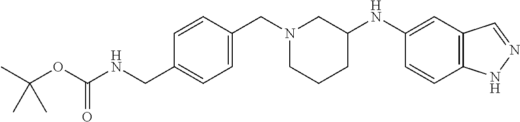 tert-butyl 4-((3-(1H-indazol-5-ylamino)piperidin-1- yl)methyl)benzylcarbamate | 1c, 7, 8, 9, 10, 13, 16c |
| 1.028 | 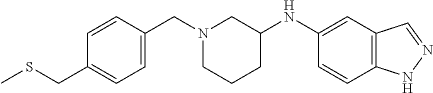 N-(1-(4-(methylthiomethyl)benzyl)piperidin-3-yl)-1H- indazol-5-amine | 1c, 7, 8, 9, 10, 13, 16c |
| 1.029 | 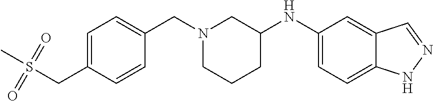 N-(1-(4-(methylsulfonylmethyl)benzyl)piperidin-3-yl)- 1H-indazol-5-amine | 1c, 7, 8, 9, 10, 13, 16c |
| 1.030 | 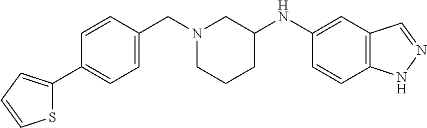 N-(1-(4-(thiophen-2-yl)benzyl)piperidin-3-yl)-1H- indazol-5-amine | 1c, 7, 8, 9, 10, 11, 14c |
| 1.031 | 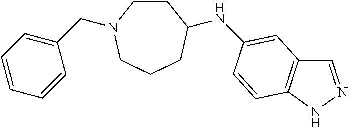 N-(1-benzylazepan-4-yl)-1H-indazol-5-amine | 1c, 7, 8, 9, 10 |
| 1.032 | 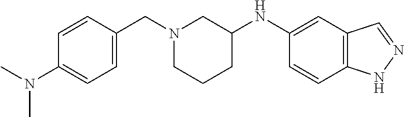 N-(1-(4-(dimethylamino)benzyl)piperidin-3-yl)-1H- indazol-5-amine | 1c, 7, 8, 9, 10 |
| 1.033 | 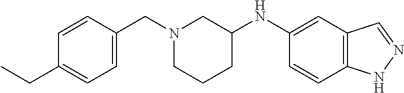 N-(1-(4-ethylbenzyl)piperidin-3-yl)-1H-indazol-5-amine | 1c, 7, 8, 9, 10 |
| 1.034 | 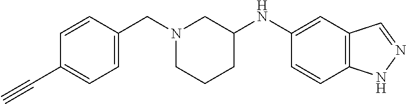 N-(1-(4-ethynylbenzyl)piperidin-3-yl)-1H-indazol-5- amine | 1c, 7, 8, 9, 10, 11, 14c |
| 1.035 | 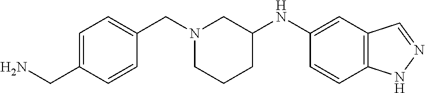 N-(1-(4-(aminomethyl)benzyl)piperidin-3-yl)-1H- indazol-5-amine | 1c, 7, 8, 9, 10, 13, 16c |
| 1.036 | 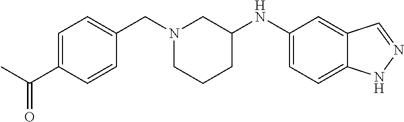 1-(4-((3-(1H-indazol-5-ylamino)piperidin-1- yl)methyl)phenyl)ethanone | 1c, 7, 8, 9, 10 |
| 1.037 | 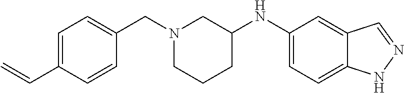 N-(1-(4-vinylbenzyl)piperidin-3-yl)-1H-indazol-5-amine | 1c, 7, 8, 9, 10, 11, 14c |
| 1.038 | 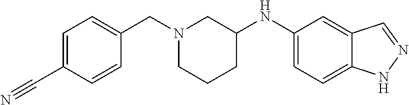 4-((3-(1H-indazol-5-ylamino)piperidin-1- yl)methyl)benzonitrile | 1c, 7, 8, 9, 10, 12, 15c |
| 1.039 | 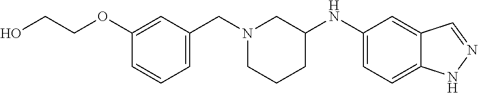 2-(3-((3-(1H-indazol-5-ylamino)piperidin-1-yl)methyl) phenoxy)ethanol | 1c, 7, 8, 9, 10, 12a, 15c, 15d |
| 1.040 | 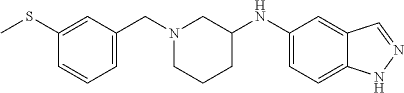 N-(1-(3-(methylthio)benzyl)piperidin-3-yl)-1H-indazol-5- amine | 1c, 7, 8, 9, 10, 12b, 15c, 15e |
| 1.041 | 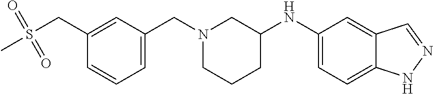 N-(1-(3-(methylsulfonylmethyl)benzyl)piperidin-3-yl)- 1H-indazol-5-amine | 1c, 7, 8, 9, 10, 13, 16c |
| 1.042 | 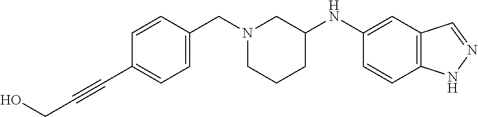 3-(4-((3-(1H-indazol-5-ylamino)piperidin-1- yl)methyl)phenyl)prop-2-yn-1-ol | 1c, 7, 8, 9, 10, 13, 16c |
| 1.043 | 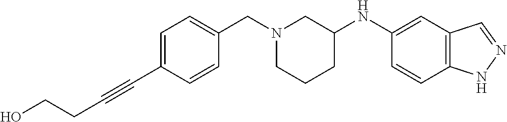 4-(4-((3-(1H-indazol-5-ylamino)piperidin-1- yl)methyl)phenyl)but-3-yn-1-ol | 1c, 7, 8, 9, 10, 13, 16c |
| 1.044 | 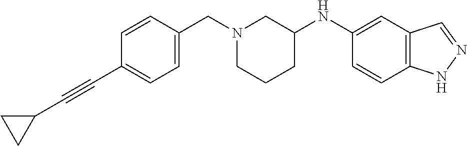 N-(1-(4-(cyclopropylethynyl)benzyl)piperidin-3-yl)-1H- indazol-5-amine | 1c, 7, 8, 9, 10, 11, 14c |
| 1.045 | 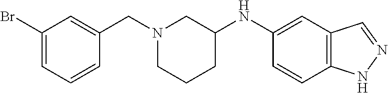 N-(1-(3-bromobenzyl)piperidin-3-yl)-1H-indazol-5- amine | 1c, 7, 8, 9,10 |
| 1.046 | 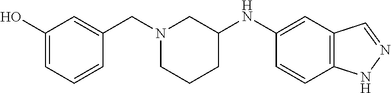 3-((3-(1H-indazol-5-ylamino)piperidin-1- yl)methyl)phenol | 1c, 7, 8, 9, 10 |
| 1.047 | 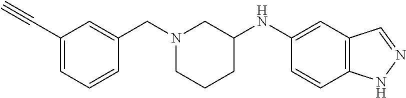 N-(1-(3-ethynylbenzyl)piperidin-3-yl)-1H-indazol-5- amine | 1c, 7, 8, 9, 10, 11, 14c |
| 1.048 | 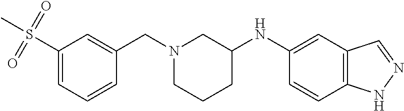 N-(1-(3-(methylsulfonyl)benzyl)piperidin-3-yl)-1H- indazol-5-amine | 1c, 7, 8, 9, 10, 12, 15c |
| 1.049 | 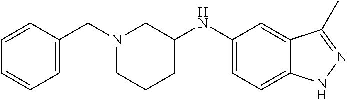 N-(1-benzylpiperidin-3-yl)-3-methyl-1H-indazol-5- amine | 1a, 6a, 8, 9, 10 |
| 1.050 | 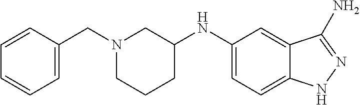 N5-(1-benzylpiperidin-3-yl)-1H-indazole-3,5-diamine | 1b, 6b, 8, 9, 10 |
| 1.051 | 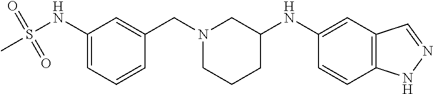 N-(3-((3-(1H-indazol-5-ylamino)piperidin-1- yl)methyl)phenyl)methanesulfonamide | 1c, 7, 8, 9, 10, 12a, 15c, 15d |
| 1.052 | 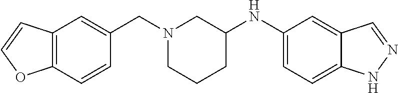 N-(1-(benzofuran-5-ylmethyl)piperidin-3-yl)-1H-indazol- 5-amine | 1c, 7, 8, 9, 10 |
| 1.053 | 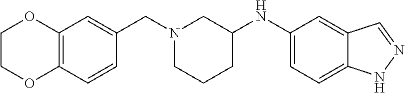 N-(1-((2,3-dihydrobenzo[b][1,4]dioxin-6- yl)methyl)piperidin-3-yl)-1H-indazol-5-amine | 1c, 7, 8, 9, 10 |
| 1.054 | 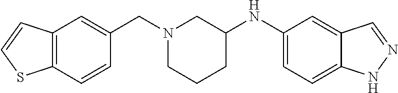 N-(1-(benzo[b]thiophen-5-ylmethyl)piperidin-3-yl)-1H- indazol-5-amine | 1c, 7, 8, 9, 10 |
| 1.055 | 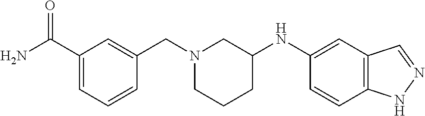 3-((3-(1H-indazol-5-ylamino)piperidin-1- yl)methyl)benzamide | 1c, 7, 8, 9, 10, 12, 15c |
| 1.056 | 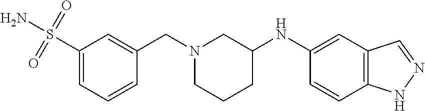 3-((3-(1H-indazol-5-ylamino)piperidin-1- yl)methyl)benzenesulfonamide | 1c, 7, 8, 9, 10, 12, 15c |
| 1.057 | 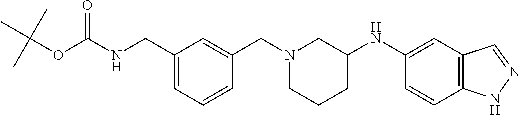 tert-butyl 3-((3-(1H-indazol-5-ylamino)piperidin-1- yl)methyl)benzylcarbamate | 1c, 7, 8, 9, 10, 13, 16c |
| 1.058 | 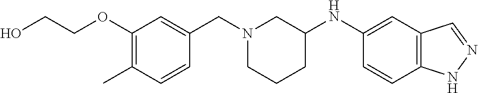 2-(5-((3-(1H-indazol-5-ylamino)piperidin-1-yl)methyl)- 2-methylphenoxy)ethanol | 1c, 7, 8, 9, 10, 12a, 15c, 15d |
| 1.059 | 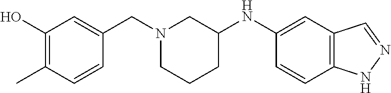 5-((3-(1H-indazol-5-ylamino)piperidin-1-yl)methyl)-2- methylphenol | 1c, 7, 8, 9, 10 |
| 1.060 | 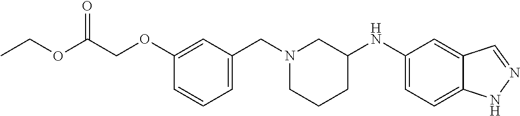 ethyl 2-(3-((3-(1H-indazol-5-ylamino)piperidin-1- yl)methyl)phenoxy)acetate | 1c, 7, 8, 9, 10, 12a, 15c, 15d |
| 1.061 | 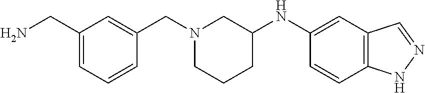 N-(1-(3-(aminomethyl)benzyl)piperidin-3-yl)-1H- indazol-5-amine | 1c, 7, 8, 9, 10, 13, 16c |
| 1.062 | 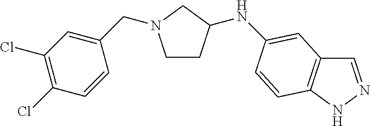 N-(1-(3,4-dichlorobenzyl)pyrrolidin-3-yl)-1H-indazol-5- amine | 1c, 7, 8, 9, 10 |
| 1.063 | 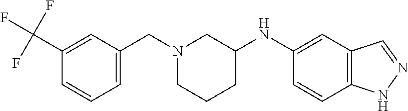 N-(1-(3-(trifluoromethyl)benzyl)piperidin-3-yl)-1H- indazol-5-amine | 1c, 7, 8, 9, 10 |
| 1.064 | 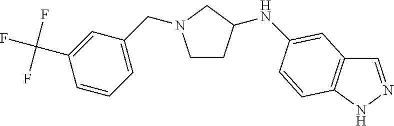 N-(1-(3-(trifluoromethyl)benzyl)pyrrolidin-3-yl)-1H- indazol-5-amine | 1c, 7, 8, 9, 10 |
| 1.065 | 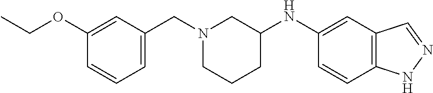 N-(1-(3-ethoxybenzyl)piperidin-3-yl)-1H-indazol-5- amine | 1c, 7, 8, 9, 10 |
| 1.066 | 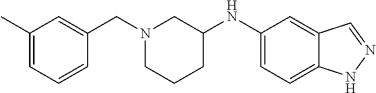 N-(1-(3-methylbenzyl)piperidin-3-yl)-1H-indazol-5- amine | 1c, 7, 8, 9, 10 |
| 1.067 | 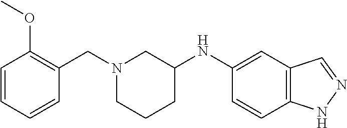 N-(1-(2-methoxybenzyl)piperidin-3-yl)-1H-indazol-5- amine | 1c, 7, 8, 9, 10 |
| 1.068 | 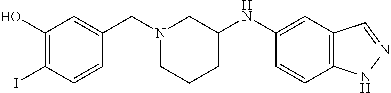 5-((3-(1H-indazol-5-ylamino)piperidin-1-yl)methyl)-2- iodophenol | 1c, 7, 8, 9, 10 |
| 1.069 | 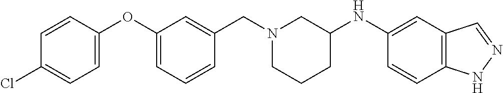 N-(1-(3-(4-chlorophenoxy)benzyl)piperidin-3-yl)-1H- indazol-5-amine | 1c, 7, 8, 9, 10 |
| 1.070 | 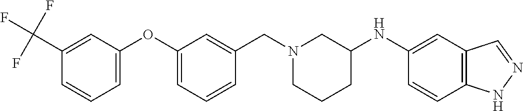 N-(1-(3-(3-(trifluoromethyl)phenoxy)benzyl)piperidin-3- yl)-1H-indazol-5-amine | 1c, 7, 8, 9, 10 |
| 1.071 | 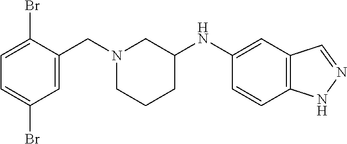 N-(1-(2,5-dibromobenzyl)piperidin-3-yl)-1H-indazol-5- amine | 1c, 7, 8, 9, 10 |
| 1.072 | 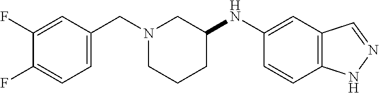 (S)-N-(1-(3,4-difluorobenzyl)piperidin-3-yl)-1H-indazol- 5-amine | 1c, 7, 8, 9, 10 |
| 1.073 | 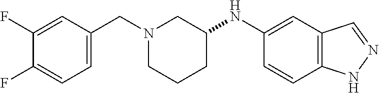 (R)-N-(1-(3,4-difluorobenzyl)piperidin-3-yl)-1H-indazol- 5-amine | 1c, 7, 8, 9, 10 |
| 1.074 | 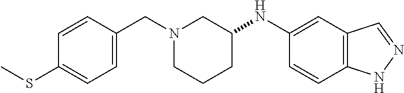 (R)-N-(1-(4-(methylthio)benzyl)piperidin-3-yl)-1H- indazol-5-amine | 1c, 7, 8, 9, 10, 12b, 15c, 15e |
| 1.075 | 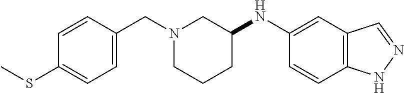 (S)-N-(1-(4-(methylthio)benzyl)piperidin-3-yl)-1H- indazol-5-amine | 1c, 7, 8, 9, 10, 12b, 15c, 15e |
| 1.076 | 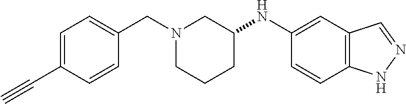 (R)-N-(1-(4-ethynylbenzyl)piperidin-3-yl)-1H-indazol-5- amine | 1c, 7, 8, 9, 10, 11, 14c |
| 1.077 | 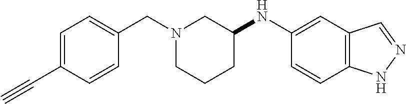 (S)-N-(1-(4-ethynylbenzyl)piperidin-3-yl)-1H-indazol-5- amine | 1c, 7, 8, 9, 10, 11, 14c |
| 1.078 | 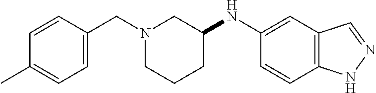 (S)-N-(1-(4-methylbenzyl)piperidin-3-yl)-1H-indazol-5- amine | 1c, 7, 8, 9, 10 |
| 1.079 | 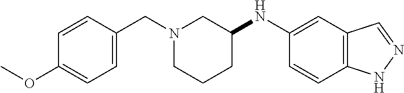 (S)-N-(1-(4-methoxybenzyl)piperidin-3-yl)-1H-indazol- 5-amine | 1c, 7, 8, 9, 10 |
| 1.080 | 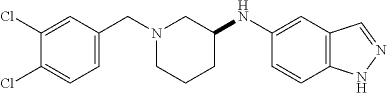 (S)-N-(1-(3,4-dichlorobenzyl)piperidin-3-yl)-1H-indazol- 5-amine | 1c, 7, 8, 9, 10 |
| 1.081 | 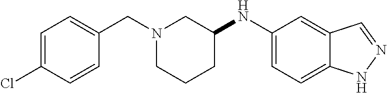 (S)-N-(1-(4-chlorobenzyl)piperidin-3-yl)-1H-indazol-5- amine | 1c, 7, 8, 9, 10 |
| 1.082 | 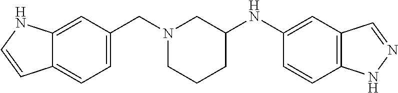 N-(1-((1H-indol-6-yl)methyl)piperidin-3-yl)-1H-indazol- 5-amine | 1c, 7, 8, 9, 10 |
| 1.083 | 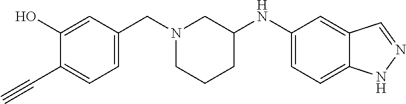 5-((3-(1H-indazol-5-ylamino)piperidin-1-yl)methyl)-2- ethynylphenol | 1c, 7, 8, 9, 10, 11, 14c |
| 1.084 | 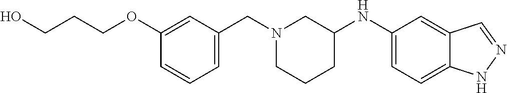 3-(3-((3-(1H-indazol-5-ylamino)piperidin-1-yl)methyl) phenoxy)propan-1-ol | 1, 7, 8, 9, 10, 12a, 15c, 15d |
| 1.085 | 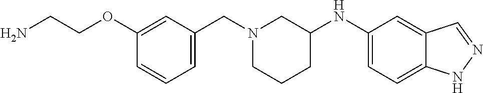 N-(1-(3-(2-aminoethoxy)benzyl)piperidin-3-yl)-1H- indazol-5-amine | 1c, 7, 8, 9, 10, 12a, 15c, 15d |
| 1.086 | 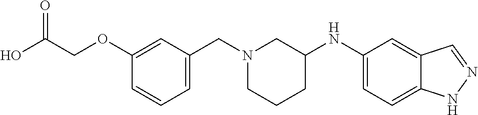 2-(3-((3-(1H-indazol-5-ylamino)piperidin-1-yl)methyl) phenoxy)acetic acid | 1c, 7, 8, 9, 10, 12a, 15c, 15d |
| 1.087 | 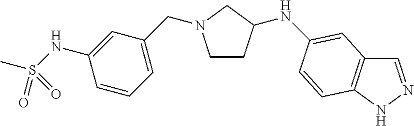 N-(3-((3-(1H-indazol-5-ylamino)pyrrolidin-1- yl)methyl)phenyl)methanesulfonamide | 1c, 7, 8, 9, 10, 12a, 15c, 15d |
| 1.088 | 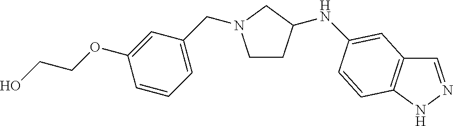 2-(3-((3-(1H-indazol-5-ylamino)pyrrolidin-1-yl)methyl) phenoxy)ethanol | 1c, 7, 8, 9, 10, 12a, 15c, 15d |
| 1.089 | 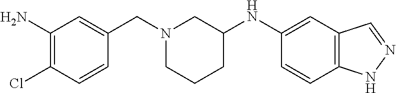 N-(1-(3-amino-4-chlorobenzyl)piperidin-3-yl)-1H- indazol-5-amine | 1c, 7, 8, 9, 10 |
| 1.090 | 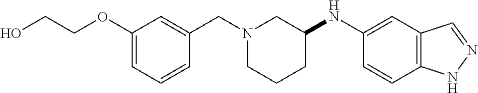 (S)-2-(3-((3-(1H-indazol-5-ylamino)piperidin-1- yl)methyl)phenoxy)ethanol | 1c, 7, 8, 9, 10, 12a, 15c, 15d |
| 1.091 | 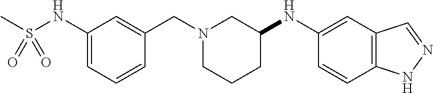 (S)-N-(3-((3-(1H-indazol-5-ylamino)piperidin-1- yl)methyl)phenyl)methanesulfonamide | 1c, 7, 8, 9, 10, 12a, 15c, 15d |
| 1.092 | 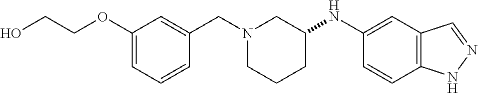 (R)-2-(3-((3-(1H-indazol-5-ylamino)piperidin-1- yl)methyl)phenoxy)ethanol | 1c, 7, 8, 9, 10, 12a, 15c, 15d |
| 1.093 | 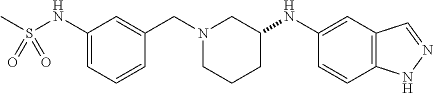 (R)-N-(3-((3-(1H-indazol-5-ylamino)piperidin-1- yl)methyl)phenyl)methanesulfonamide | 1c, 7, 8, 9, 10, 12a, 15c, 15d |
| 1.094 | 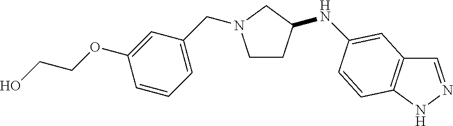 (S)-2-(3-((3-(1H-indazol-5-ylamino)pyrrolidin-1- yl)methyl)phenoxy)ethanol | 1c, 7, 8, 9, 10, 12a, 15c, 15d |
| 1.095 | 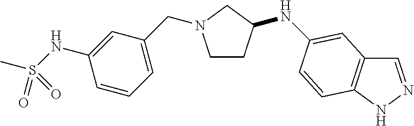 (S)-N-(3-((3-(1H-indazol-5-ylamino)pyrrolidin-1- yl)methyl)phenyl)methanesulfonamide | 1c, 7, 8, 9, 10, 12a, 15c, 15d |
| 1.096 | 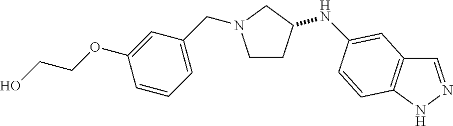 (R)-2-(3-((3-(1H-indazol-5-ylamino)pyrrolidin-1- yl)methyl)phenoxy)ethanol | 1c, 7, 8, 9, 10, 12a, 15c, 15d |
| 1.097 | 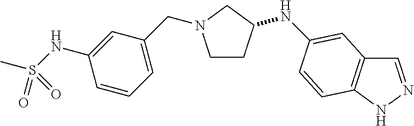 (R)-N-(3-((3-(1H-indazol-5-ylamino)pyrrolidin-1- yl)methyl)phenyl)methanesulfonamide | 1c, 7, 8, 9, 10, 12a, 15c, 15d |
| 1.098 | 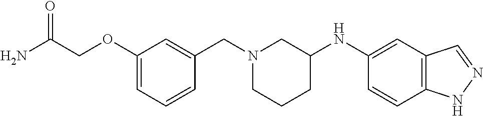 2-(3-((3-(1H-indazol-5-ylamino)piperidin-1- yl)methyl)phenoxy)acetamide | 1c, 7, 8, 9, 10, 12a, 15c, 15d |
| 1.099 | 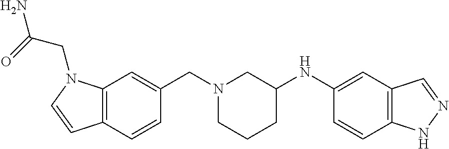 2-(6-((3-(1H-indazol-5-ylamino)piperidin-1-yl)methyl)- 1H-indol-1-yl)acetamide | 1c, 7, 8, 9, 10, 13, 16c |
| 1.100 | 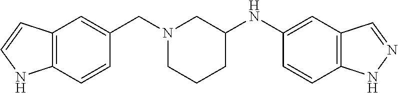 N-(1-((1H-indol-5-yl)methyl)piperidin-3-yl)-1H-indazol- 5-amine | 1c, 7, 8, 9, 10, 13, 16c |
| 1.101 | 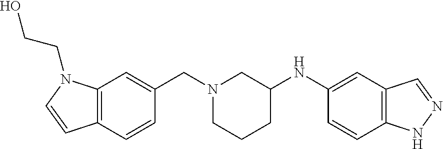 2-(6-((3-(1H-indazol-5-ylamino)piperidin-1-yl)methyl)- 1H-indol-1-yl)ethanol | 1c, 7, 8, 9, 10, 13, 16c |
| 1.102 | 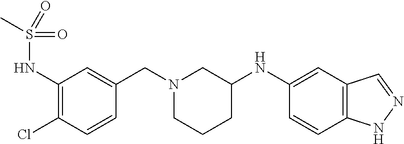 N-(5-((3-(1H-indazol-5-ylamino)piperidin-1-yl)methyl)- 2-chlorophenyl)methanesulfonamide | 1c, 7, 8, 9, 10, 12a, 15c, 15d |
| 1.103 | 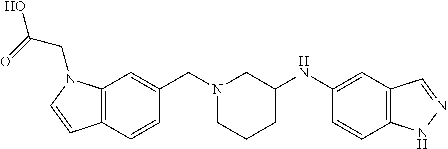 2-(6-((3-(1H-indazol-5-ylamino)piperidin-1-yl)methyl)- 1H-indol-1-yl)acetic acid | 1c, 7, 8, 9, 10, 13, 16c |
| 1.104 | 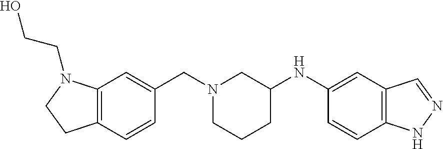 2-(6-((3-(1H-indazol-5-ylamino)piperidin-1- yl)methyl)indolin-1-yl)ethanol | 1c, 7, 8, 9, 10, 13, 16c |
| 1.105 | 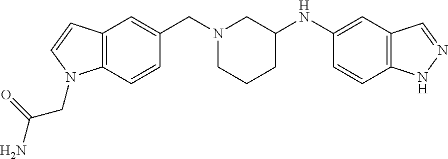 2-(5-((3-(1H-indazol-5-ylamino)piperidin-1-yl)methyl)- 1H-indol-1-yl)acetamide | 1c, 7, 8, 9, 10, 13, 16c |
| 1.106 | 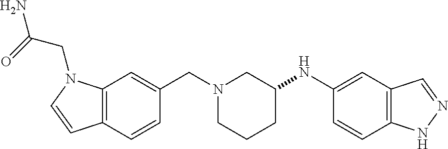 (R)-2-(6-((3-(1H-indazol-5-ylamino)piperidin-1- yl)methyl)-1H-indol-1-yl)acetamide | 1c, 7, 8, 9, 10, 13, 16c |
| 1.107 | 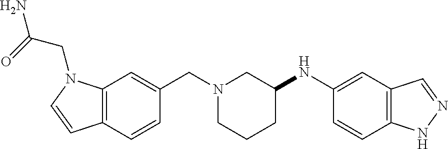 (S)-2-(6-((3-(1H-indazol-5-ylamino)piperidin-1- yl)methyl)-1H-indol-1-yl)acetamide | 1c, 7, 8, 9, 10, 13, 16c |
| 1.108 | 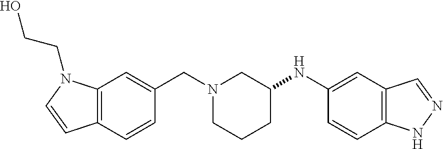 (R)-2-(6-((3-(1H-indazol-5-ylamino)piperidin-1- yl)methyl)-1H-indol-1-yl)ethanol | 1c, 7, 8, 9, 10, 13, 16c |
| 1.109 | 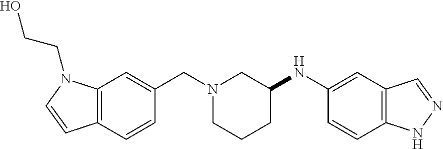 (S)-2-(6-((3-(1H-indazol-5-ylamino)piperidin-1- yl)methyl)-1H-indol-1-yl)ethanol | 1c, 7, 8, 9, 10, 13, 16c |
| 1.110 | 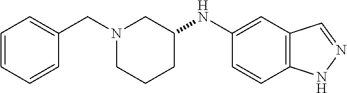 (R)-N-(1-benzylpiperidin-3-yl)-1H-indazol-5-amine | 1c, 7, 8, 9, 10 |
| 1.111 | 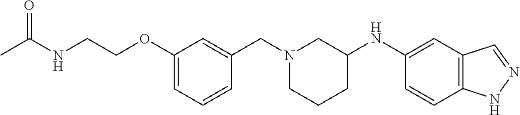 N-(2-(3-((3-(1H-indazol-5-ylamino)piperidin-1- yl)methyl)phenoxy)ethyl)acetamide | 1c, 7, 8, 9, 10, 12a, 15c, 15d |
| 1.112 | 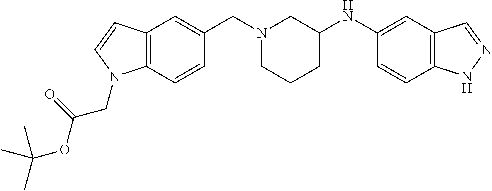 tert-butyl 2-(5-((3-(1H-indazol-5-ylamino)piperidin-1- yl)methyl)-1H-indol-1-yl)acetate | 1c, 7, 8, 9, 10, 13, 16c |
| 1.113 | 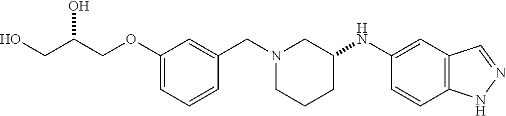 (S)-3-(3-(((R)-3-(1H-indazol-5-ylamino)piperidin-1- yl)methyl)phenoxy)propane-1,2-diol | 1c, 7, 8, 9, 10, 12a, 15c, 15d |
| 1.114 | 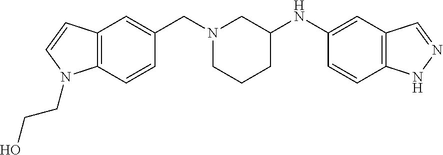 2-(5-((3-(1H-indazol-5-ylamino)piperidin-1-yl)methyl)- 1H-indol-1-yl)ethanol | 1c, 7, 8, 9, 10, 13, 16c |
| 1.115 | 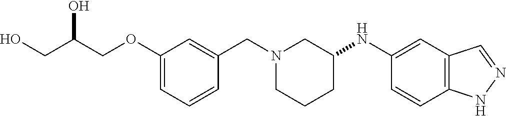 (R)-3-(3-(((R)-3-(1H-indazol-5-ylamino)piperidin-1- yl)methyl)phenoxy)propane-1,2-diol | 1c, 7, 8, 9, 10, 12a, 15c, 15d |
| 1.116 | 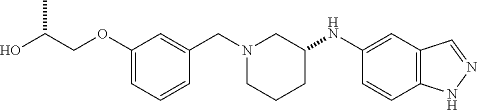 (R)-1-(3-(((R)-3-(1H-indazol-5-ylamino)piperidin-1- yl)methyl)phenoxy)propan-2-ol | 1c, 7, 8, 9, 10, 12a, 15c, 15d |
| 1.117 | 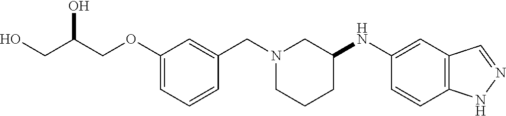 (R)-3-(3-(((S)-3-(1H-indazol-5-ylamino)piperidin-1- yl)methyl)phenoxy)propane-1,2-diol | 1c, 7, 8, 9, 10, 12a, 15c, 15d |
| 1.118 | 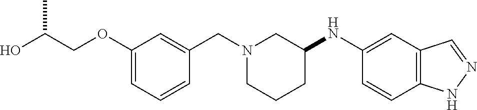 (R)-1-(3-(((S)-3-(1H-indazol-5-ylamino)piperidin-1- yl)methyl)phenoxy)propan-2-ol | 1c, 7, 8, 9, 10, 12a, 15c, 15d |
| 1.119 | 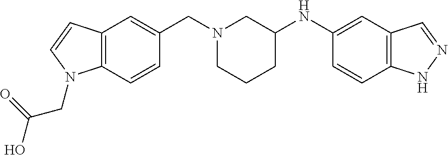 2-(5-((3-(1H-indazol-5-ylamino)piperidin-1-yl)methyl)- 1H-indol-1-yl)acetic acid | 1c, 7, 8, 9, 10, 13, 16c |
| 1.120 | 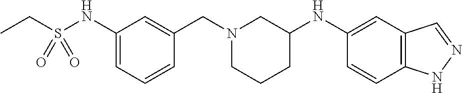 N-(3-((3-(1H-indazol-5-ylamino)piperidin-1- yl)methyl)phenyl)ethanesulfonamide | 1c, 7, 8, 9, 10, 12a, 15c, 15d |
| 1.121 | 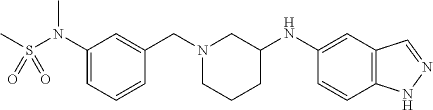 N-(3-((3-(1H-indazol-5-ylamino)piperidin-1- yl)methyl)phenyl)-N-methylmethanesulfonamide | 1c, 7, 8, 9, 10, 12a, 15c, 15d |
| 1.122 | 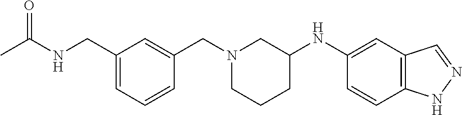 N-(3-((3-(1H-indazol-5-ylamino)piperidin-1- yl)methyl)benzyl)acetamide | 1c, 7, 8, 9, 10, 13, 16c |
| 1.123 | 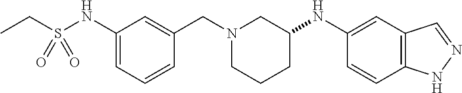 (R)-N-(3-((3-(1H-indazol-5-ylamino)piperidin-1- yl)methyl)phenyl)ethanesulfonamide | 1c, 7, 8, 9, 10, 12a, 15c, 15d |
| 1.124 | 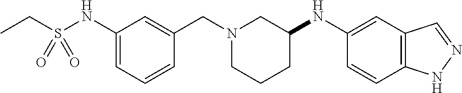 (S)-N-(3-((3-(1H-indazol-5-ylamino)piperidin-1- yl)methyl)phenyl)ethanesulfonamide | 1c, 7, 8, 9, 10, 12a, 15c, 15d |
| 1.125 | 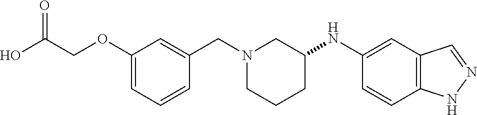 (R)-2-(3-((3-(1H-indazol-5-ylamino)piperidin-1- yl)methyl)phenoxy)acetic acid | 1c, 7, 8, 9, 10, 12a, 15c, 15d |
| 1.126 | 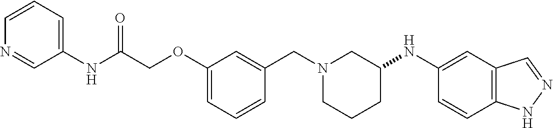 (R)-2-(3-((3-(1H-indazol-5-ylamino)piperidin-1- yl)methyl)phenoxy)-N-(pyridin-3-yl)acetamide | 1c, 7, 8, 9, 10, 12a, 15c, 15d |
| 1.127 | 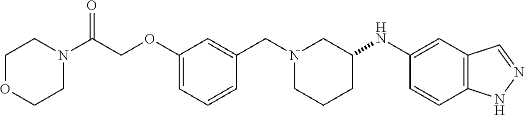 (R)-2-(3-((3-(1H-indazol-5-ylamino)piperidin-1- yl)methyl)phenoxy)-1-morpholinoethanone | 1c, 7, 8, 9, 10, 12a, 15c, 15d |
| 1.128 | 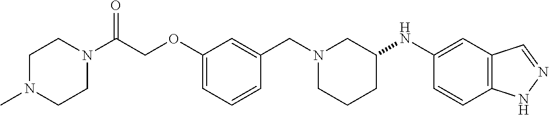 (R)-2-(3-((3-(1H-indazol-5-ylamino)piperidin-1- yl)methyl)phenoxy)-1-(4-methylpiperazin-1-yl)ethanone | 1c, 7, 8, 9, 10, 12a, 15c, 15d |
| 1.129 | 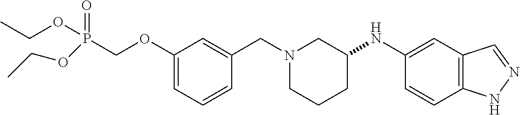 (R)-diethyl (3-((3-(1H-indazol-5-ylamino)piperidin-1- yl)methyl)phenoxy)methylphosphonate | 1c, 7, 8, 9, 10, 12a, 15c, 15d |
| 1.130 | 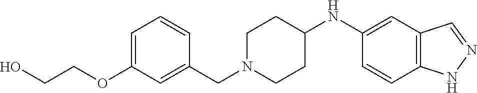 2-(3-((4-(1H-indazol-5-ylamino)piperidin-1- yl)methyl)phenoxy)ethanol | 1c, 7, 8, 9, 10, 12a, 15c, 15d |
| 1.131 | 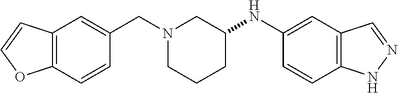 (R)-N-(1-(benzofuran-5-ylmethyl)piperidin-3-yl)-1H- indazol-5-amine | 1c, 7, 8, 9, 10 |
| 1.132 | 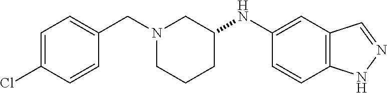 (R)-N-(1-(4-chlorobenzyl)piperidin-3-yl)-1H-indazol-5- amine | 1c, 7, 8, 9, 10 |
| 1.133 | 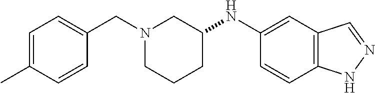 (R)-N-(1-(4-methylbenzyl)piperidin-3-yl)-1H-indazol-5- amine | 1c, 7, 8, 9, 10 |
| 1.134 | 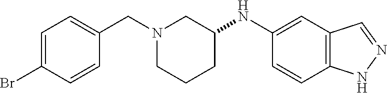 (R)-N-(1-(4-bromobenzyl)piperidin-3-yl)-1H-indazol-5- amine | 1c, 7, 8, 9, 10 |
| 1.136 | 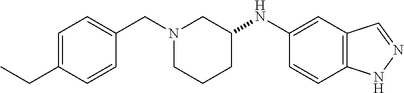 (R)-N-(1-(4-ethylbenzyl)piperidin-3-yl)-1H-indazol-5- amine | 1c, 7, 8, 9, 10 |
| 1.137 | 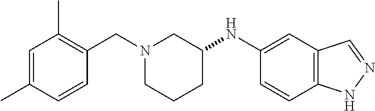 (R)-N-(1-(2,4-dimethylbenzyl)piperidin-3-yl)-1H- indazol-5-amine | 1c, 7, 8, 9, 10 |
| 1.138 | 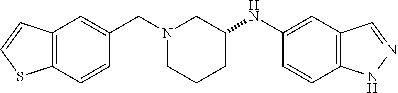 (R)-N-(1-(benzo[b]thiophen-5-ylmethyl)piperidin-3-yl)- 1H-indazol-5-amine | 1c, 7, 8, 9, 10 |
| 1.139 | 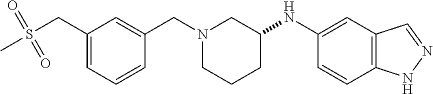 (R)-N-(1-(3-(methylsulfonylmethyl)benzyl)piperidin-3- yl)-1H-indazol-5-amine | 1c, 7, 8, 9, 10, 12, 15c |
| 1.140 | 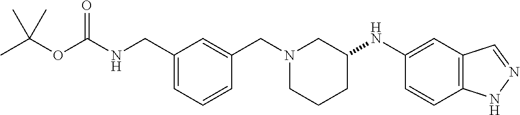 (R)-tert-butyl 1H-indazol-5-ylamino)piperidin-1- yl)methyl)benzylcarbamate | 1c, 7, 8, 9, 10, 13, 16c |
| 1.141 | 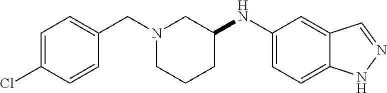 (S)-N-(1-(4-chlorobenzyl)piperidin-3-yl)-1H-indazol-5- amine | 1c, 7, 8, 9, 10 |
| 1.142 | 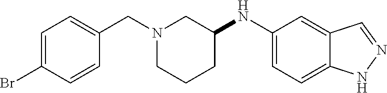 (S)-N-(1-(4-bromobenzyl)piperidin-3-yl)-1H-indazol-5- amine | 1c, 7, 8, 9, 10 |
| 1.143 | 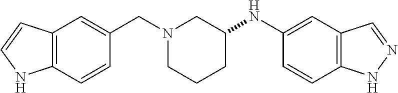 (R)-N-(1-((1H-indol-5-yl)methyl)piperidin-3-yl)-1H- indazol-5-amine | 1c, 7, 8, 9, 10, 13, 16c |
| 1.144 | 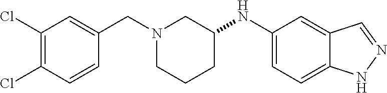 (R)-N-(1-(3,4-dichlorobenzyl)piperidin-3-yl)-1H-indazol- 5-amine | 1c, 7, 8, 9, 10 |
| 1.145 | 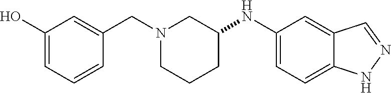 (R)-3-((3-(1H-indazol-5-ylamino)piperidin-1- yl)methyl)phenol | 1c, 7, 8, 9, 10 |
| 1.146 | 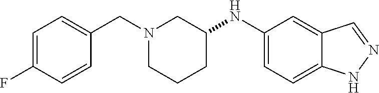 (R)-N-(1-(4-fluorobenzyl)piperidin-3-yl)-1H-indazol-5- amine | 1c, 7, 8, 9, 10 |
| 1.147 | 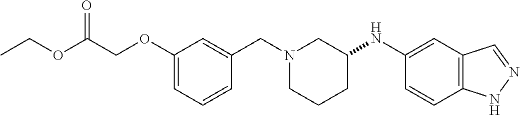 (R)-ethyl 2-(3-((3-(1H-indazol-5-ylamino)piperidin-1- yl)methyl)phenoxy)acetate | 1c, 7, 8, 9, 10, 12a, 15c, 15d |
| 1.148 | 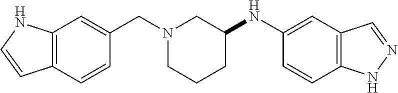 (S)-N-(1-((1H-indol-6-yl)methyl)piperidin-3-yl)-1H- indazol-5-amine | 1c, 7, 8, 9, 10, 13, 16c |
| 1.149 | 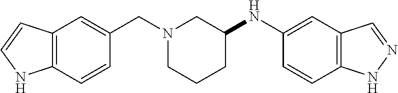 (S)-N-(1-((1H-indol-5-yl)methyl)piperidin-3-yl)-1H- indazol-5-amine | 1c, 7, 8, 9, 10, 13, 16c |
| 1.150 | 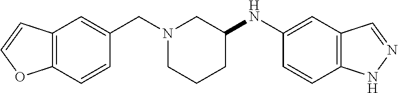 (S)-N-(1-(benzofuran-5-ylmethyl)piperidin-3-yl)-1H- indazol-5-amine | 1c, 7, 8, 9, 10 |
| 1.151 | 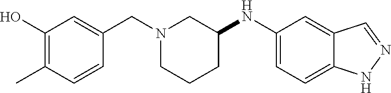 (S)-5-((3-(1H-indazol-5-ylamino)piperidin-1-yl)methyl)- 2-methylphenol | 1c, 7, 8, 9, 10 |
| 1.152 | 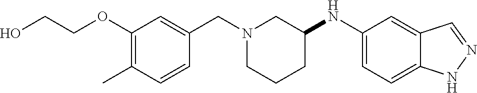 (S)-2-(5-((3-(1H-indazol-5-ylamino)piperidin-1- yl)methyl)-2-methylphenoxy)ethanol | 1c, 7, 8, 9, 10, 12a, 15c, 15d |
| 1.153 | 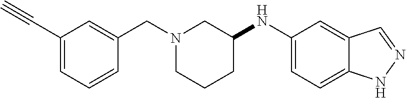 (S)-N-(1-(3-ethynylbenzyl)piperidin-3-yl)-1H-indazol-5- amine | 1c, 7, 8, 9, 10, 11, 14c |
| 1.154 | 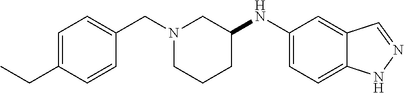 (S)-N-(1-(4-ethylbenzyl)piperidin-3-yl)-1H-indazol-5- amine | 1c, 7, 8, 9, 10 |
| 1.155 | 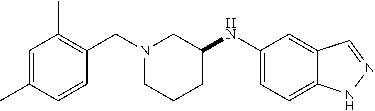 (S)-N-(1-(2,4-dimethylbenzyl)piperidin-3-yl)-1H- indazol-5-amine | 1c, 7, 8, 9, 10 |
| 1.156 | 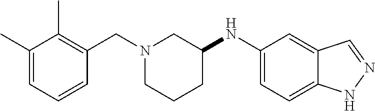 (S)-N-(1-(2,3-dimethylbenzyl)piperidin-3-yl)-1H- indazol-5-amine | 1c, 7, 8, 9, 10 |
| 1.157 | 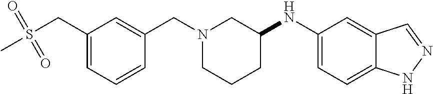 (S)-N-(1-(3-(methylsulfonylmethyl)benzyl)piperidin-3- yl)-1H-indazol-5-amine | 1c, 7, 8, 9, 10, 12, 15c |
| 1.158 | 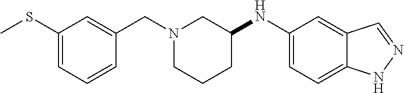 (S)-N-(1-(3-(methylthio)benzyl)piperidin-3-yl)-1H- indazol-5-amine | 1c, 7, 8, 9, 10, 12b, 15c, 15e |
| 1.159 | 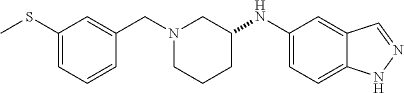 (R)-N-(1-(3-(methylthio)benzyl)piperidin-3-yl)-1H- indazol-5-amine | 1c, 7, 8, 9, 10, 12b, 15c, 15e |
| 1.160 | 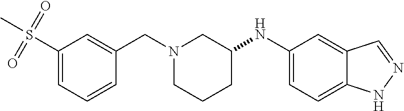 (R)-N-(1-(3-(methylsulfonyl)benzyl)piperidin-3-yl)-1H- indazol-5-amine | 1c, 7, 8, 9, 10, 12, 15c |
| 1.161 | 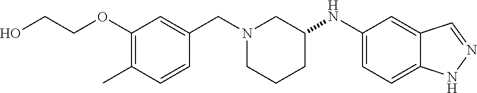 (R)-2-(5-((3-(1H-indazol-5-ylamino)piperidin-1- yl)methyl)-2-methylphenoxy)ethanol | 1c, 7, 8, 9, 10, 12a, 15c, 15d |
| 1.162 | 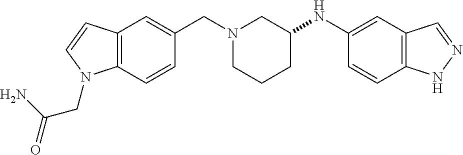 (R)-2-(5-((3-(1H-indazol-5-ylamino)piperidin-1- yl)methyl)-1H-indol-1-yl)acetamide | 1c, 7, 8, 9, 10, 13, 16c |
| 1.163 | 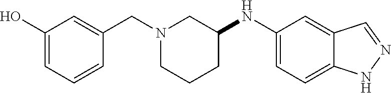 (S)-3-((3-(1H-indazol-5-ylamino)piperidin-1- yl)methyl)phenol | 1c, 7, 8, 9, 10 |
| 1.164 | 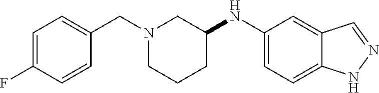 (S)-N-(1-(4-fluorobenzyl)piperidin-3-yl)-1H-indazol-5- amine | 1c, 7, 8, 9, 10 |
| 1.165 | 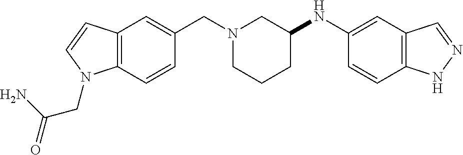 (S)-2-(5-((3-(1H-indazol-5-ylamino)piperidin-1- yl)methyl)-1H-indol-1-yl)acetamide | 1c, 7, 8, 9, 10, 13, 16c |
| 1.166 | 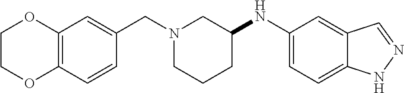 (S)-N-(1-((2,3-dihydrobenzo[b][1,4]dioxin-6- yl)methyl)piperidin-3-yl)-1H-indazol-5-amine | 1c, 7, 8, 9, 10 |
| 1.167 | 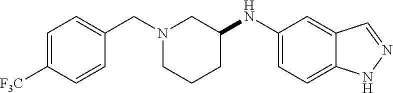 (S)-N-(1-(4-(trifluoromethyl)benzyl)piperidin-3-yl)-1H- indazol-5-amine | 1c, 7, 8, 9, 10 |
| 1.168 | 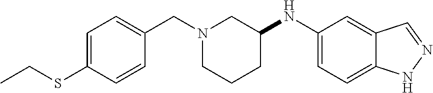 (S)-N-(1-(4-(ethylthio)benzyl)piperidin-3-yl)-1H-indazol- 5-amine | 1c, 7, 8, 9, 10, 12b, 15c, 15e |
| 1.169 | 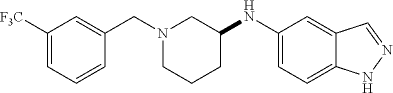 (S)-N-(1-(3-(trifluoromethyl)benzyl)piperidin-3-yl)-1H- indazol-5-amine | 1c, 7, 8, 9, 10 |
| 1.170 | 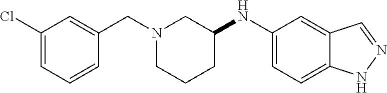 (S)-N-(1-(3-chlorobenzyl)piperidin-3-yl)-1H-indazol-5- amine | 1c, 7, 8, 9, 10 |
| 1.171 | 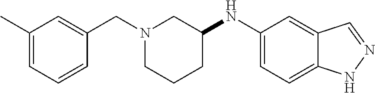 (S)-N-(1-(3-methylbenzyl)piperidin-3-yl)-1H-indazol-5- amine | 1.171 |
| 1.172 | 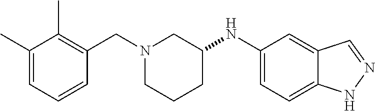 (R)-N-(1-(2,3-dimethylbenzyl)piperidin-3-yl)-1H- indazol-5-amine | 1.172 |
| 1.173 | 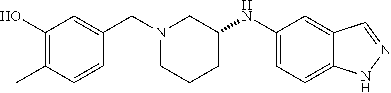 (R)-5-((3-(1H-indazol-5-ylamino)piperidin-1-yl)methyl)- 2-methylphenol | 1.173 |
| 1.174 | 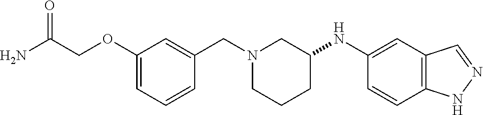 (R)-2-(3-((3-(1H-indazol-5-ylamino)piperidin-1- yl)methyl)phenoxy)acetamide | 1.174 |
| 1.175 | 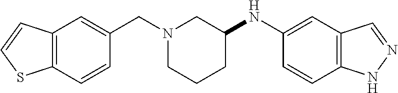 (S)-N-(1-(benzo[b]thiophen-5-ylmethyl)piperidin-3-yl)- 1H-indazol-5-amine | 1.175 |
| 1.176 | 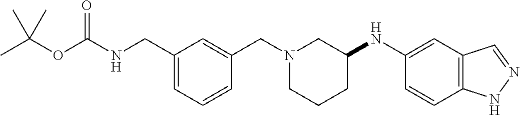 (S)-tert-butyl 1H-indazol-5-ylamino)piperidin-1- yl)methyl)benzylcarbamate | 1.176 |
| 1.177 | 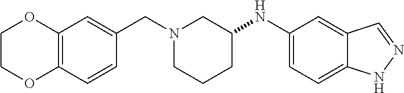 (R)-N-(1-((2,3-dihydrobenzo[b][1,4]dioxin-6- yl)methyl)piperidin-3-yl)-1H-indazol-5-amine | 1.177 |
| 1.178 | 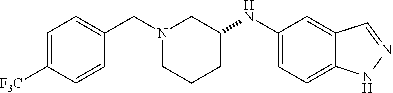 (R)-N-(1-(4-(trifluoromethyl)benzyl)piperidin-3-yl)-1H- indazol-5-amine | 1.178 |
| 1.179 | 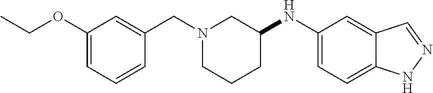 (S)-N-(1-(3-ethoxybenzyl)piperidin-3-yl)-1H-indazol-5- amine | 1.179 |
| 1.180 | 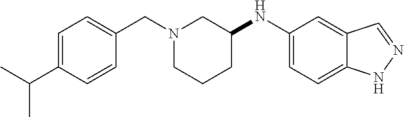 (S)-N-(1-(4-isopropylbenzyl)piperidin-3-yl)-1H-indazol- 5-amine | 1.180 |
| 1.181 | 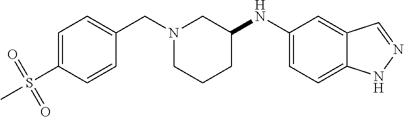 (S)-N-(1-(4-(methylsulfonyl)benzyl)piperidin-3-yl)-1H- indazol-5-amine | 1.181 |
| 1.182 | 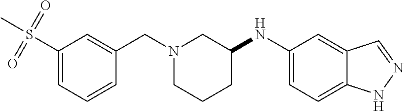 (S)-N-(1-(3-(methylsulfonyl)benzyl)piperidin-3-yl)-1H- indazol-5-amine | 1.182 |
| 1.183 | 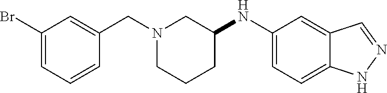 (S)-N-(1-(3-bromobenzyl)piperidin-3-yl)-1H-indazol-5- amine | 1.183 |
| 1.184 | 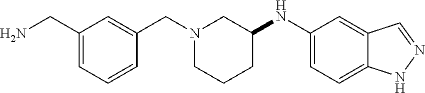 (S)-N-(1-(3-(aminomethyl)benzyl)piperidin-3-yl)-1H- indazol-5-amine | 1.184 |
| 1.185 | 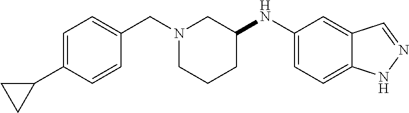 (S)-N-(1-(4-cyclopropylbenzyl)piperidin-3-yl)-1H- indazol-5-amine | 1.185 |
| 1.186 | 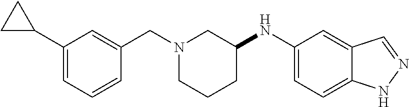 (S)-N-(1-(3-cyclopropylbenzyl)piperidin-3-yl)-1H- indazol-5-amine | 1.186 |
| 1.187 | 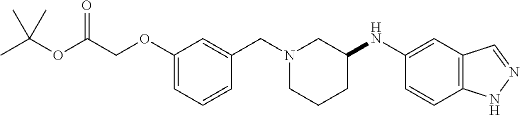 (S)-tert-butyl 2-(3-((3-(1H-indazol-5-ylamino)piperidin- 1-yl)methyl)phenoxy)acetate | 1.187 |
| 1.188 | 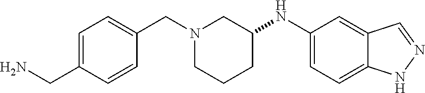 (R)-N-(1-(4-(aminomethyl)benzyl)piperidin-3-yl)-1H- indazol-5-amine | 1.188 |
| 1.189 | 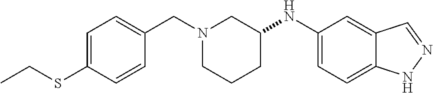 (R)-N-(1-(4-(ethylthio)benzyl)piperidin-3-yl)-1H-indazol- 5-amine | 1.189 |
| 1.190 | 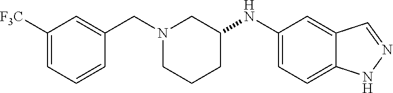 (R)-N-(1-(3-(trifluoromethyl)benzyl)piperidin-3-yl)-1H- indazol-5-amine | 1.190 |
| 1.191 | 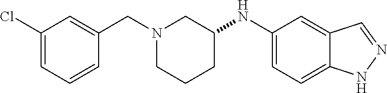 (R)-N-(1-(3-chlorobenzyl)piperldin-3-yl)-1H-indazol-5- amine | 1c, 7, 8, 9, 10 |
| 1.192 | 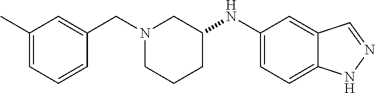 (R)-N-(1-(3-methylbenzyl)piperidin-3-yl)-1H-indazol-5- amine | 1c, 7, 8, 9, 10 |
| 1.193 | 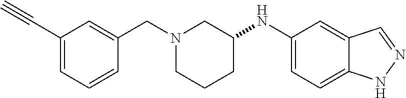 (R)-N-(1-(3-ethynylbenzyl)piperidin-3-yl)-1H-indazol-5- amine | 1c, 7, 8, 9, 10, 11, 14c |
| 1.194 | 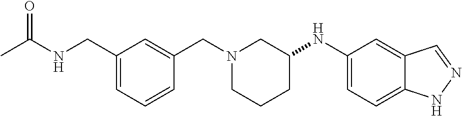 (R)-N-(1H-indazol-5-ylamino)piperidin-1- yl)methyl)benzyl)acetamide | 1c, 7, 8, 9, 10, 13, 16c |
| 1.195 | 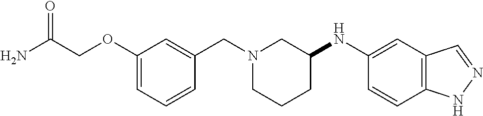 (S)-2-(3-((3-(1H-indazol-5-ylamino)piperidin-1- yl)methyl)phenyl1H-indazol-5-ylamino)piperidin-1- yl)methyl)phenoxy)acetamide | 1c, 7, 8, 9, 10, 12a, 15c, 15d |
| 1.196 | 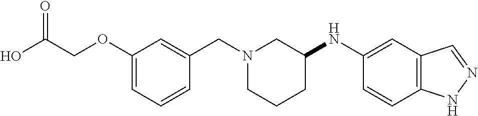 (S)-2-(3-((3-(1H-indazol-5-ylamino)piperidin-1- yl)methyl)phenoxy)acetic acid | 1c 7, 8, 9, 10, 12a, 15c, 15d |
| 1.197 | 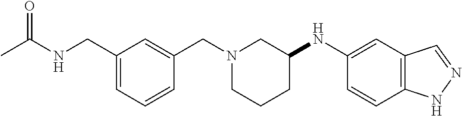 (S)-N-(1H-indazol-5-ylamino)piperidin-1- yl)methyl)benzyl)acetamide | 1c, 7, 8, 9, 10, 13, 16c |
| 1.198 | 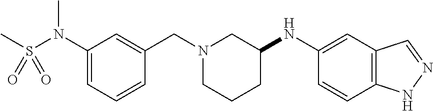 (S)-N-(3-((3-(1H-indazol-5-ylamino)piperidin-1- yl)methyl)phenyl)-N-methylmethanesulfonamide | 1c, 7, 8, 9, 10, 12a, 15c, 15d |
| 1.199 | 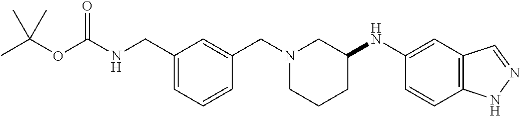 (S)-tert-butyl 1H-indazol-5-ylamino)piperidin-1- yl)methyl)benzylcarbamate | 1c, 7, 8, 9, 10, 13, 16c |
| 1.200 | 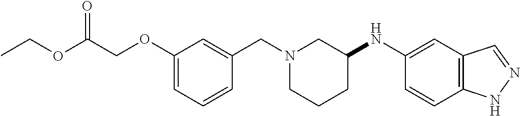 (3)-ethyl 2-(3-((3-(1H-indazol-5-ylamino)piperidin-1- yl)methyl)phenoxy)acetate | 1c, 7, 8, 9, 10, 12a, 15c, 15d |
| 1.201 | 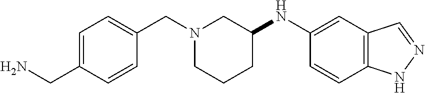 (S)-N-(1-(4-(aminomethyl)benzyl)piperidin-3-yl)-1H- indazol-5-amine | 1c, 7, 8, 9, 10, 13, 16c |
| 1.202 | 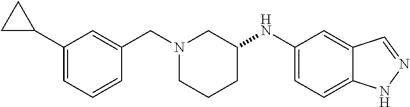 (R)-N-(1-(3-cyclopropylbenzyl)piperidin-3-yl)-1H- indazol-6-amine | 1c, 7, 8, 9, 10, 11, 14c |
| 1.203 | 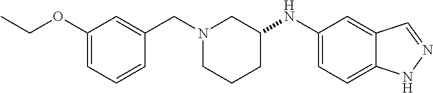 (R)-N-(1-(3-ethoxybenzyl)piperidin-3-yl)-1H-indazol-5- amine | 1c, 7, 8, 9, 10 |
| 1.204 | 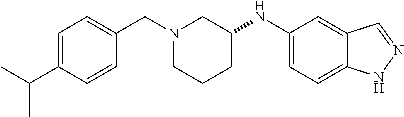 (R)-N-(1-(4-isopropylbenzyl)piperidin-3-yl)-1H-indazol- 5-amine | 1c, 7, 8, 9, 10 |
| 1.205 | 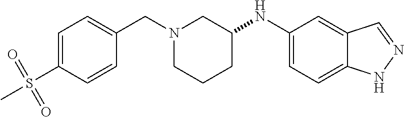 (R)-N-(1-(4-(methylsulfonyl)benzyl)piperidin-3-yl)-1H- indazol-5-amine | 1c, 7, 8, 9, 10, 12, 15c |
| 1.206 | 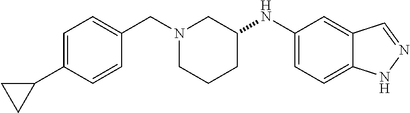 (R)-N-(1-(4-cyclopropylbenzyl)piperidin-3-yl)-1H- indazol-5-amine | 1c, 7, 8, 9, 10, 11, 14c |
| 1.207 | 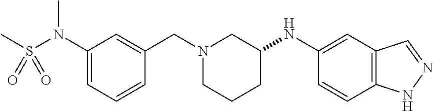 (R)-N-(3-((3-(1H-indazol-5-ylamino)piperidin-1- yl)methyl)phenyl)-N-methylmethanesulfonamide | 1c, 7, 8, 9, 10, 12a, 15c, 15d |
| 1.208 | 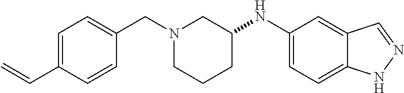 (R)-N-(1-(4-vinylbenzyl)piperidin-3-yl)-1H-indazol-5- amine | 1c, 7, 8, 9, 10, 11, 14c |
| 1.209 | 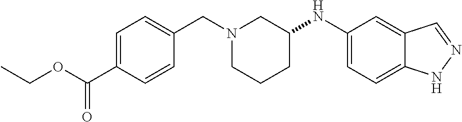 (R)-ethyl 4-((3-(1H-indazol-5-ylamino)piperidin-1- yl)methyl)benzoate | 1c, 7, 8, 9, 10 |
| 1.210 | 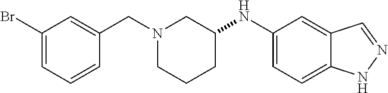 (R)-N-(1-(3-bromobenzyl)piperidin-3-yl)-1H-indazol-5- amine | 1c, 7, 8, 9, 10 |
| 1.211 | 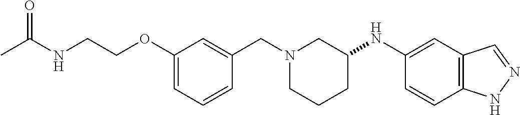 (R)-N-(2-(3-((3-(1H-indazol-5-ylamino)piperidin-1- yl)methyl)phenoxy)ethyl)acetamide | 1c, 7, 8, 9, 10, 12a, 15c, 15d |
| 1.212 | 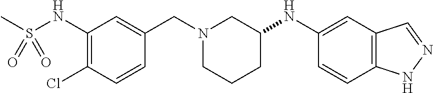 (R)-N-(5-((3-(1H-indazol-5-ylamino)piperidin-1- yl)methyl)-2-chlorophenyl)methanesulfonamide | 1c, 7, 8, 9, 10, 12a, 15c, 15d |
| 1.213 | 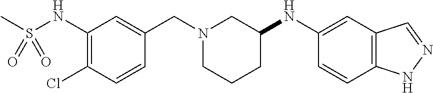 (S)-N-(5-((3-(1H-indazol-5-ylamino)piperidin-1- yl)methyl)-2-chlorophenyl)methanesulfonamide | 1c, 7, 8, 9, 10, 12a, 15c, 15d |
| 1.214 | 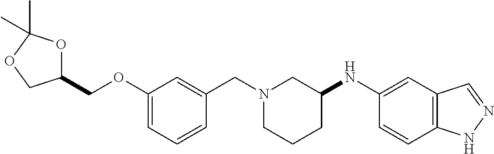 N-((S)-1-(3-(((S)-2,2-dimethyl-1,3-dioxolan-4- yl)methoxy)benzyl)piperidin-3-yl)-1H-indazol-5-amine | 1c, 7, 8, 9, 10, 12a, 15c, 15d |
| 1.215 | 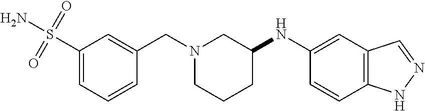 (S)-3-((3-(1H-indazol-5-ylamino)piperidin-1- yl)methyl)benzenesulfonamide | 1c, 7, 8, 9, 10, 12, 15c |
| 1.216 | 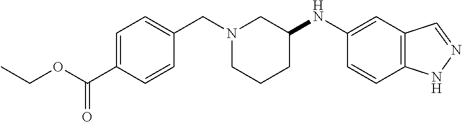 (S)-ethyl 4-((3-(1H-indazol-5-ylamino)piperidin-1- yl)methyl)benzoate | 1c, 7, 8, 9, 10 |
| 1.217 | 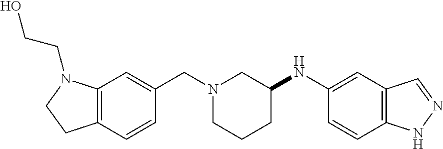 (S)-2-(6-((3-(1H-indazol-5-ylamino)piperidin-1- yl)methyl)indolin-1-yl)ethanol | 1c, 7, 8, 9, 10, 13, 16c |
| 1.218 | 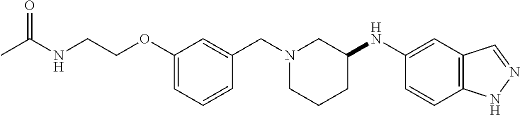 (S)-N-(2-(3-((3-(1H-indazol-5-ylamino)piperidin-1- yl)methyl)phenoxy)ethyl)acetamide | 1c, 7, 8, 9, 10, 12a, 15c, 15d |
| 1.219 | 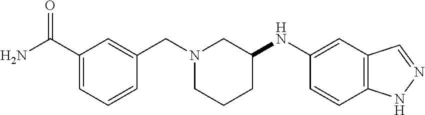 (S)-3-((3-(1H-indazol-5-ylamino)piperidin-1- yl)methyl)benzamide | 1c, 7, 8, 9, 10, 12, 15c |
| 1.220 | 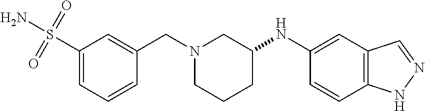 (R)-3-((3-(1H-indazol-5-ylamino)piperidin-1- yl)methyl)benzenesulfonamide | 1c, 7, 8, 9, 10, 12, 15c |
| 1.221 | 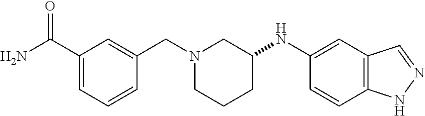 (R)-3-((3-(1H-indazol-5-ylamino)piperidin-1- yl)methyl)benzamide | 1c, 7, 8, 9, 10, 12, 15c |
| 1.222 | 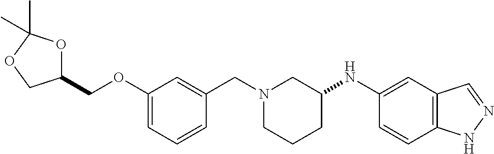 N-((R)-1-(3-(((S)-2,2-dimethyl-1,3-dioxolan-4- yl)methoxy)benzyl)piperidin-3-yl)-1H-indazol-5-amine | 1c, 7, 8, 9, 10, 12a, 15c, 15d |
| 1.223 | 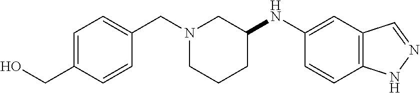 (S)-(4-((3-(1H-indazol-5-ylamino)piperidin-1- yl)methyl)phenyl)methanol | 1c, 7, 8, 9, 10 |
| 1.224 | 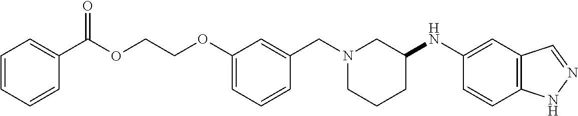 (S)-2-(3-((3-(1H-indazol-5-ylamino)piperidin-1- yl)methyl)phenoxy)ethyl benzoate | 1c, 7, 8, 9, 10, 12a, 15c, 15d |
| 1.225 | 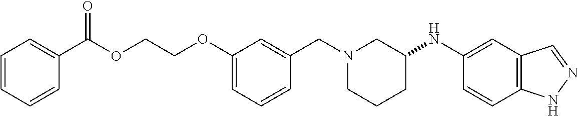 (R)-2-(3-((3-(1H-indazol-5-ylamino)piperidin-1- yl)methyl)phenoxy)ethyl benzoate | 1c, 7, 8, 9, 10, 12a, 15c, 15d |
| 1.226 | 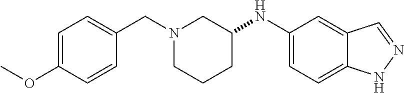 (R)-N-(1-(4-methoxybenzyl)piperidin-3-yl)-1H-indazol- 5-amine | 1c, 7, 8, 9, 10 |
| 1.227 | 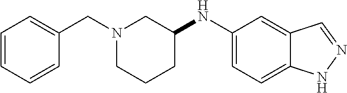 (S)-N-(1-benzylpiperidin-3-yl)-1H-indazol-5-amine | 1c, 7, 8, 9, 10 |
| 1.228 | 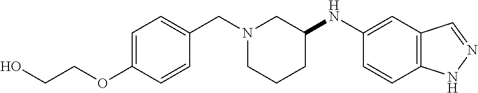 (S)-2-(4-((3-(1H-indazol-5-ylamino)piperidin-1- yl)methyl)phenoxy)ethanol | 1c, 7, 8, 9,10, 12a, 15c, 15d |
| 1.229 | 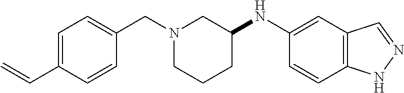 (S)-N-(1-(4-vinylbenzyl)piperidin-3-yl)-1H-indazol-5- amine | 1c, 7, 8, 9, 10, 11, 14c |
| 1.230 | 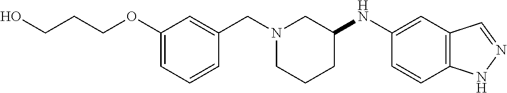 (S)-3-(3-((3-(1H-indazol-5-ylamino)piperidin-1- yl)methyl)phenoxy)propan-1-ol | 1c, 7, 8, 9, 10, 12a, 15c, 15d |
| 1.231 | 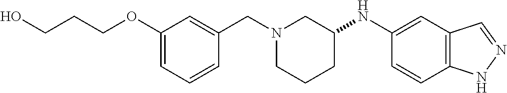 (R)-3-(3-((3-(1H-indazol-5-ylamino)piperidin-1- yl)methyl)phenoxy)propan-1-ol | 1c, 7, 8, 9, 10, 12a, 15c, 15d |
| 1.232 | 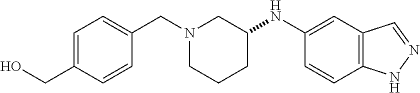 (R)-(4-((3-(1H-indazol-5-ylamino)piperidin-1- yl)methyl)phenyl)methanol | 1c, 7, 8, 9, 10 |
| 1.233 | 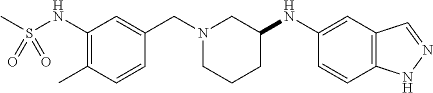 (S)-N-(5-((3-(1H-indazol-5-ylamino)piperidin-1- yl)methyl)-2-methylphenyl)methanesulfonamide | 1c, 7, 8, 9, 10, 12a, 15c, 15d |
| 1.234 | 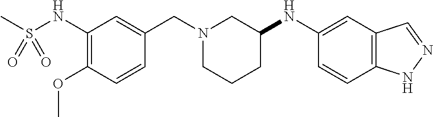 (S)-N-(5-((3-(1H-indazol-5-ylamino)piperidin-1- yl)methyl)-2-methoxyphenyl)methanesulfonamide | 1c, 7, 8, 9, 10, 12a 15c, 15d |
| 1.235 | 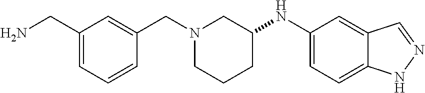 (R)-N-(1-(3-(aminomethyl)benzyl)piperidin-3-yl)-1H- indazol-5-amine | 1c, 7, 8, 9, 10, 13, 16c |
| 2.001 | 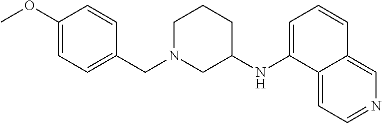 N-(1-(4-methoxybenzyl)piperidin-3-yl)isoquinolin-5- amine | 2c, 7, 8, 9, 10 |
| 2.002 | 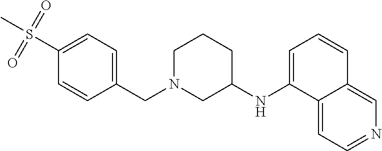 N-(1-(4-(methylsulfonyl)benzyl)piperidin-3- yl)isoquinolin-5-amine | 2c, 7, 8, 9, 10, 12, 15c |
| 2.003 | 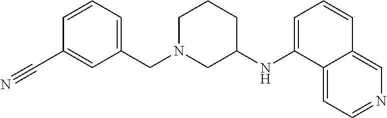 3-((3-(isoquinolin-5-ylamino)piperidin-1- yl)methyl)benzonitrile | 2c, 7, 8, 9, 10, 12, 15c |
| 2.004 | 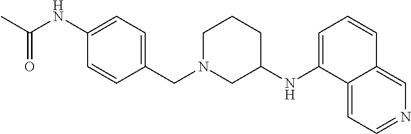 N-(4-((3-(isoquinolin-5-ylamino)piperidin-1- yl)methyl)phenyl)acetamide | 2c, 7, 8, 9, 10, 12a, 15c, 15d |
| 2.005 | 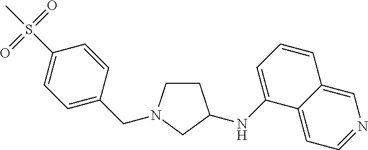 N-(1-(4-(methylsulfonyl)benzyl)pyrrolidin-3- yl)isoquinolin-5-amine | 2c, 7, 8, 9, 10, 12, 15c |
| 2.006 | 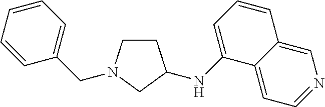 N-(1-benzylpyrrolidin-3-yl)isoquinolin-5-amine | 2c, 7, 8, 9, 10 |
| 2.007 | 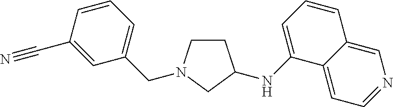 3-((3-(isoquinolin-5-ylamino)pyrrolidin-1- yl)methyl)benzonitrile | 2c, 7, 8, 9, 10, 12, 15c |
| 2.008 | 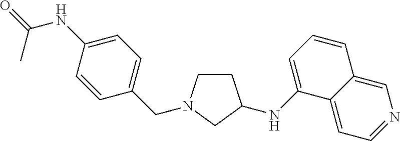 N-(4-((3-(isoquinolin-5-ylamino)pyrrolidin-1- yl)methyl)phenyl)acetamide | 2c, 7, 8, 9, 10, 12a, 15c, 15d |
| 2.009 | 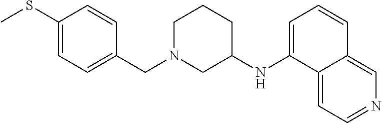 N-(1-(4-(methylthio)benzyl)piperidin-3-yl)isoquinolin-5- amine | 2c, 7, 8, 9, 10, 12b, 15c, 15e |
| 2.010 | 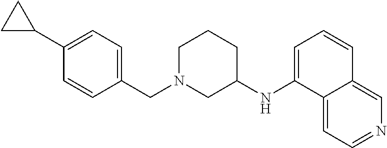 N-(1-(4-cyclopropylbenzyl)pperidin-3-yl)isoquinolin-5- amine | 2c, 7, 8, 9, 10, 11, 14c |
| 2.011 | 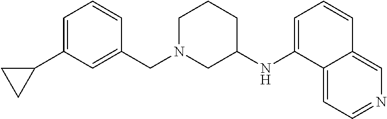 N-(1-(3-cyclopropylbenzyl)piperidin-3-yl)isoquinolin-5- amine | 2c, 7, 8, 9, 10, 11, 14c |
| 2.012 | 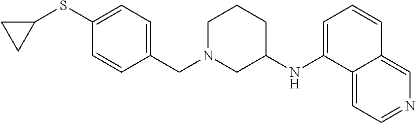 N-(1-(4-(cyclopropylthio)benzyl)piperidin-3- yl)isoquinolin-5-amine | 2c, 7, 8, 9, 10, 12b, 15c, 15e |
| 2.013 | 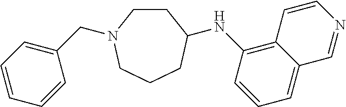 N-(1-benzylazepan-4-yl)isoquinolin-5-amine | 2c, 7, 8, 9, 10 |
| 2.014 | 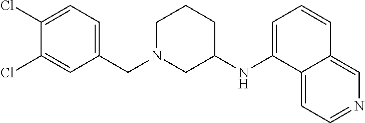 N-(1-(3,4-dichlorobenzyl)piperidin-3-yl)isoquinolin-5- amine | 2c, 7, 8, 9, 10 |
| 2.015 | 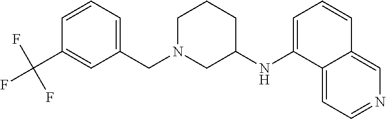 N-(1-(3-(trifluoromethyl)benzyl)piperidin-3- yl)isoquinolin-5-amine | 2c, 7, 8, 9, 10 |
| 2.016 | 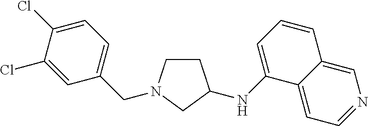 N-(1-(3,4-dichlorobenzyl)pyrrolidin-3-yl)isoquinolin-5- amine | 2c, 7, 8, 9, 10 |
| 2.017 | 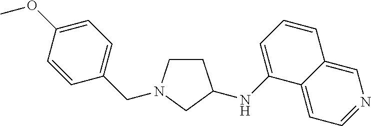 N-(1-(4-methoxybenzyl)pyrrolidin-3-yl)isoquinolin-5- amine | 2c, 7, 8, 9, 10 |
| 2.018 | 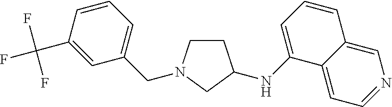 N-(1-(3-(trifluoromethyl)benzyl)pyrrolidin-3- yl)isoquinolin-5-amine | 2c, 7, 8, 9, 10 |
| 2.019 | 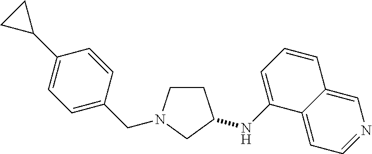 (S)-N-(1-(4-cyclopropylbenzyl)pyrrolidin-3- yl)isoquinolin-5-amine | 2c, 7, 8, 9, 10, 11, 14c |
| 2.020 | 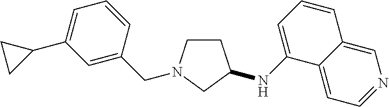 (R)-N-(1-(3-cyclopropylbenzyl)pyrrolidin-3- yl)isoquinolin-5-amine | 2c, 7, 8, 9, 10, 11, 14c |
| 2.021 | 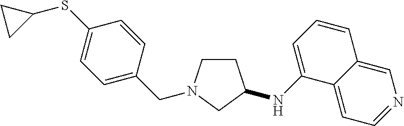 (R)-N-(1-(4-(cyclopropylthio)benzyl)pyrrolidin-3- yl)isoquinolin-5-amine | 2c, 7, 8, 9, 10, 12b, 15c, 15e |
| 2.022 | 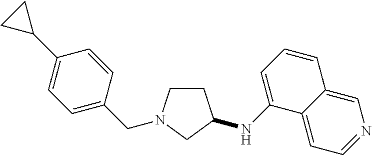 (R)-N-(1-(4-cyclopropylbenzyl)pyrrolidin-3- yl)isoquinolin-5-amine | 2c, 7, 8, 9, 10, 11, 14c |
| 2.023 | 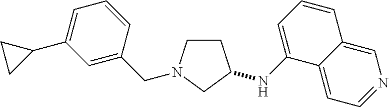 (S)-N-(1-(3-cyclopropylbenzyl)pyrrolidin-3- y1)isoquinolin-5-amine | 2c, 7, 8, 9, 10, 11, 14c |
| 2.024 | 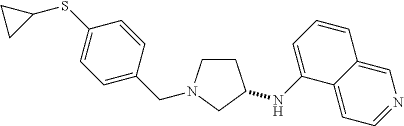 (S)-N-(1-(4-(cyclopropylthio)benzyl)pyrrolidin-3- yl)isoquinolin-5-amine | 2c, 7, 8, 9, 10, 12b, 15c, 15e |
| 2.025 | 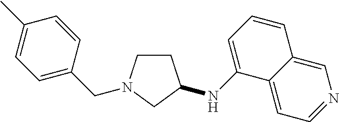 (R)-N-(1-(4-methylbenzyl)pyrrolidin-3-yl)isoquinolin-5- amine | 2c, 7, 8, 9, 10 |
| 2.026 | 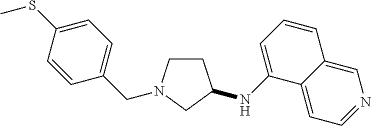 (R)-N-(1-(4-(methylthio)benzyl)pyrrolidin-3- y1)isoquinolin-5-amine | 2c, 7, 8, 9, 10, 12b, 15c, 15e |
| 2.027 | 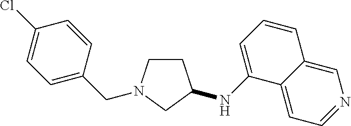 (R)-N-(1-(4-chlorobenzyl)pyrrolidin-3-yl)isoquinolin-5- amine | 2c, 7, 8, 9, 10 |
| 2.028 | 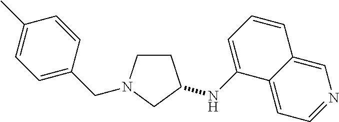 (S)-N-(1-(4-methylbenzyl)pyrrolidin-3-yl)isoquinolin-5- amine | 2c, 7, 8, 9, 10 |
| 2.029 | 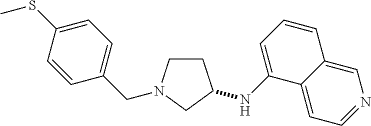 (S)-N-(1-(4-(methylthio)benzyl)pyrrolidin-3- yl)isoquinolin-5-amine | 2c, 7, 8, 9, 10, 12b, 15c, 15e |
| 2.030 | 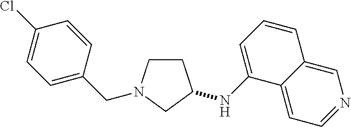 (S)-N-(1-(4-chlorobenzyl)pyrrolidin-3-yl)isoquinolin-5- amine | 2c, 7, 8, 9, 10 |
| 2.031 | 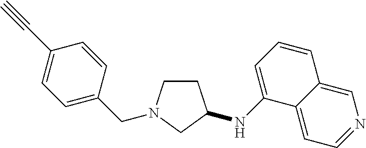 (R)-N-(1-(4-ethynylbenzyl)pyrrolidin-3-yl)isoquinolin-5- amine | 2c, 7, 8, 9, 10, 11, 14c |
| 2.032 | 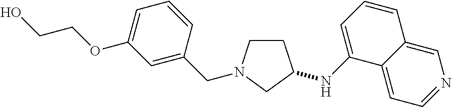 (S)-2-(3-((3-(isoquinolin-5-ylamino)pyrrolidin-1- yl)methyl)phenoxy)ethanol | 2c, 7, 8, 9, 10, 12a 15c, 15d |
| 2.033 | 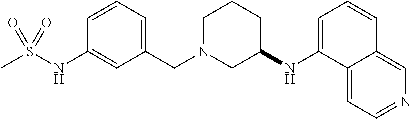 (R)-N-(3-((3-(isoquinolin-5-ylamino)piperidin-1- yl)methyl)phenyl)methanesulfonamide | 2c, 7, 8, 9, 10, 12a, 15c, 15d |
| 2.034 | 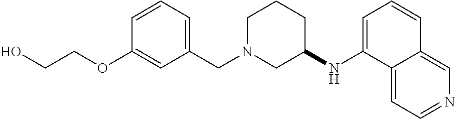 (R)-2-(3-((3-(isoquinolin-5-ylamino)piperidin-1- yl)methyl)phenoxy)ethanol | 2c, 7, 8, 9, 10, 12a, 15c, 15d |
| 2.035 | 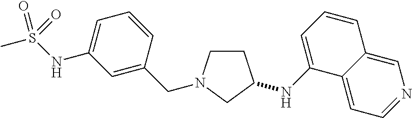 (S)-N-(3-((3-(isoquinolin-5-ylamino)pyrrolidin-1- yl)methyl)phenyl)methanesulfonamide | 2c, 7, 8, 9, 10, 12a, 15c, 15d |
| 2.036 | 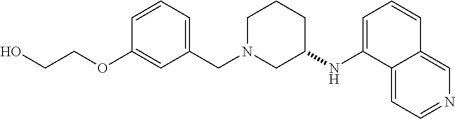 (S)-2-(3-((3-(isoquinolin-5-ylamino)piperidin-1- yl)methyl)phenoxy)ethanol | 2c, 7, 8, 9, 10, 12a, 15c, 15d |
| 2.037 | 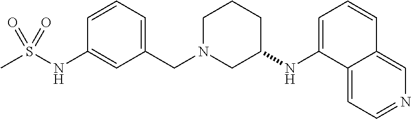 (S)-N-(3-((3-(isoquinolin-5-ylamino)piperidin-1- yl)methyl)phenyl)methanesulfonamide | 2c, 7, 8, 9, 10, 12a, 15c, 15d |
| 2.038 | 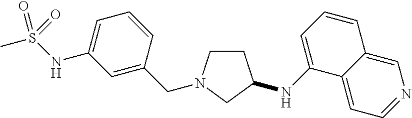 (R)-N-(3-((3-(isoquinolin-5-ylamino)pyrrolidin-1- yl)methyl)phenyl)methanesulfonamide | 2c, 7, 8, 9, 10, 12a, 15c, 15d |
| 2.039 | 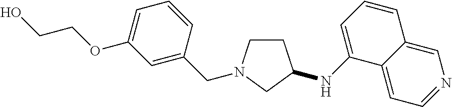 (R)-2-(3-((3-(isoquinolin-5-ylamino)pyrrolidin-1- yl)methyl)phenoxy)ethanol | 2c, 7, 8, 9, 10, 12a, 15c, 15d |
| 2.040 | 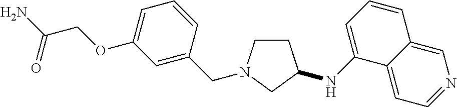 (R)-2-(3-((3-(isoquinolin-5-ylamino)pyrrolidin-1- yl)methyl)phenoxy)acetamide | 2c, 7, 8, 9, 10, 12a, 15c, 15d |
| 2.041 | 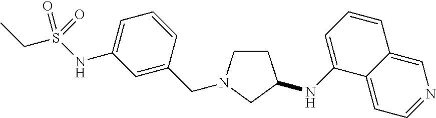 (R)-N-(3-((3-(isoquinolin-5-ylamino)pyrrolidin-1- yl)methyl)phenyl)ethanesulfonamide | 2c, 7, 8, 9, 10, 12a, 15c, 15d |
| 2.042 | 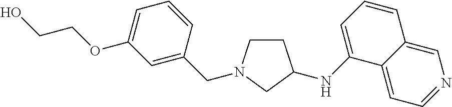 2-(3-((3-(isoquinolin-5-ylamino)pyrrolidin-1- yl)methyl)phenoxy)ethanol | 2c, 7, 8, 9, 10, 12a, 15c, 15d |
| 2.043 | 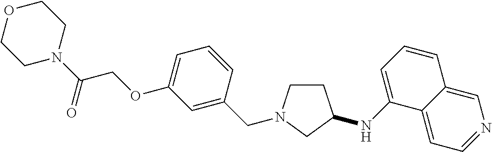 (R)-2-(3-((3-(isoquinolin-5-ylamino)pyrrolidin-1- yl)methyl)phenoxy)-1-morpholinoethanone | 2c, 7, 8, 9, 10, 12a, 15c, 15d |
| 2.044 | 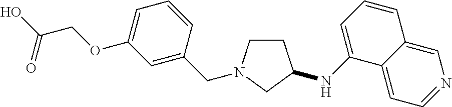 (R)-2-(3-((3-(isoquinolin-5-ylamino)pyrrolidin-1- yl)methyl)phenoxy)acetic acid | 2c, 7, 8, 9, 10, 12a, 15c, 15d |
| 2.045 | 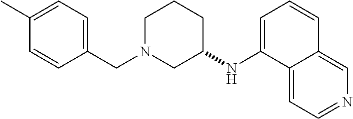 (S)-N-(1-(4-methylbenzyl)piperidin-3-yl)isoquinolin-5- amine | 2c, 7, 8, 9, 10 |
| 2.046 | 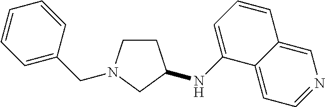 (R)-N-(1-benzylpyrrolidin-3-yl)isoquinolin-5-amine | 2c, 7, 8, 9, 10 |
| 2.047 | 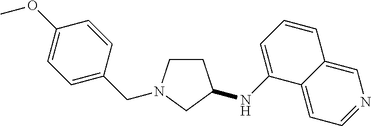 (R)-N-(1-(4-methoxybenzyl)pyrrolidin-3-yl)isoquinolin- 5-amine | 2c, 7, 8, 9, 10 |
| 2.048 | 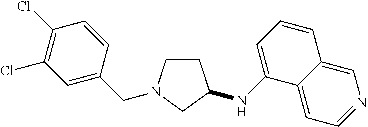 (R)-N-(1-(3,4-dichlorobenzyl)pyrrolidin-3-yl)isoquinolin- 5-amine | 2c, 7, 8, 9, 10 |
| 2.049 | 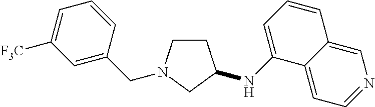 (R)-N-(1-(3-(trifluoromethyl)benzyl)pyrrolidin-3- yl)isoquinolin-5-amine | 2c, 7, 8, 9, 10 |
| 2.050 | 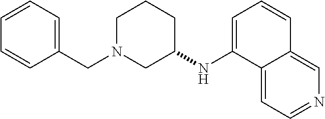 (S)-N-(1-benzylpiperidin-3-yl)isoquinolin-5-amine | 2c, 7, 8, 9, 10 |
| 2.051 | 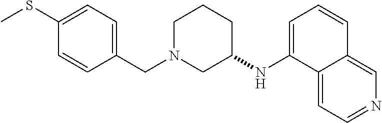 (S)-N-(1-(4-(methylthio)benzyl)piperidin-3- yl)isoquinolin-5-amine | 2c, 7, 8, 9, 10, 12b, 15c, 15e |
| 2.052 | 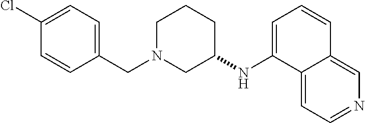 (S)-N-(1-(4-chlorobenzyl)piperidin-3-yl)isoquinolin-5- amine | 2c, 7, 8, 9, 10 |
| 2.053 | 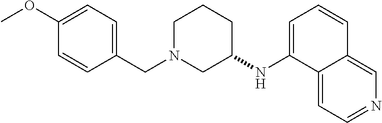 (S)-N-(1-(4-methoxybenzyl)piperidin-3-yl)isoquinolin-5- amine | 2c, 7, 8, 9, 10 |
| 3.001 | 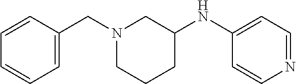 N-(1-benzylpiperidin-3-yl)pyridin-4-amine | 3c, 7, 8, 9, 10 |
| 3.002 | 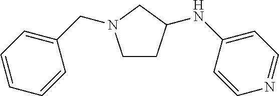 N-(1-benzylpyrrolidin-3-yl)pyridin-4-amine | 3c, 7, 8, 9, 10 |
| 4.001 | 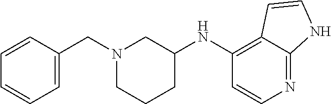 N-(1-benzylpiperidin-3-yl)-1H-pyrrolo[2,3-b]pyridin-4- amine | 4c, 7, 8, 9, 10 |
| 4.002 | 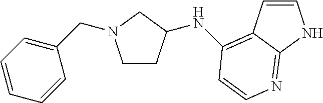 N-(1-benzylpyrrolidin-3-yl)-1H-pyrrolo[2,3-b]pyridin-4- amine | 4c, 7, 8, 9, 10 |
| 5.001 | 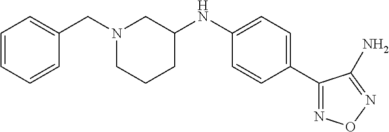 4-(4-(1-benzylpiperidin-3-ylamino)phenyl)-1,2,5- oxadiazol-3-amine | 5a, 7, 8, 9, 10 |
| 5.002 | 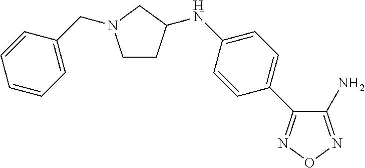 4-(4-(1-benzylpyrrolidin-3-ylamino)phenyl)-1,2,5- oxadiazol-3-amine | 5a, 7, 8, 9, 10 |

Preferred ROCK inhibitor compounds of this invention include, but are not limited to the ROCK inhibitor compounds of embodiments 5, 14, 15, and 16 as described above, and their associated salts, tautomers, solvates, or hydrates. Compound 2.039 and Compound 1.123 are particularly preferred.

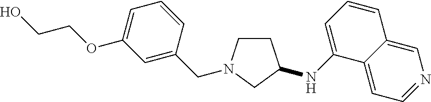

### (R)-2-(3-((3-(isoquinolin-5-ylamino)pyrrolidin-1-yl)methyl)phenoxy)ethanol (Compound 2.039)

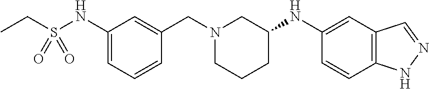

### (R)—N-(3-((3-(1H-indazol-5-ylamino)piperidin-1-yl)methyl)phenyl)ethanesulfonamide (Compound 1.123)

### Pharmaceutical Formulation

This invention provides a formulation containing one or more agents that enhance the ophthalmic properties of ROCK inhibitor compounds formulated in an aqueous medium whose pH is adjusted to enhance ocular surface residence time and the bioavailability in the aqueous humor of the anterior chamber, and to reduce systemic exposure.

The invention provides an aqueous formulation of ROCK inhibitor compound(s) that is suitable for therapeutic use and remains stable under normal use storage conditions for an extended period of time. The formulation is useful on lowering intraocular pressure in mammals. For topical administration, one to two drops of these formulations are delivered to the surface of the eye one to four times per day.

The aqueous ophthalmic formulations of this invention have an increased residence time on the ocular surface and/or aqueous humor concentrations without a concomitant increase in systemic concentrations.

The present invention is directed to an aqueous pharmaceutical formulation comprising 0.001-2% ROCK inhibitor compound, 1-100 mM buffer suitable to maintain the pH about 6.3-7.8, 0.01-2% surfactant, and a tonicity agent to maintain a tonicity about 220-360 mOsm/kG. “About” as used herein, refers to ±15%. Preferred pH is about 6.3-7.5, and more preferred pH is about 6.3-7.3,

The concentration of ROCK inhibitor compound in the aqueous formulation is in general 0.001-2%, preferably 0.01-0.5%, more preferably 0.01-0.4%, more preferably 0.03-0.2%, and more prefereably 0.03-0.15, or 0.03-0.1% (w/v).

Buffers suitable to maintain the pH between 6.3 and 7.8 include citrate, phosphate, maleate, or combination thereof. Suitable concentration of the buffer is 1-10 mM, preferably 5-50 mM, more preferably 5-25 mM, and most preferably 10-20 mM.

Surfactants (surface active agents) or solubilizing agents suitable for the present invention are those acceptable for use in ophthalmic preparations. The surfactants can be ionic or non-ionic. Preferably, this surfactant is non-ionic. Useful surfactants include but are not limited to polysorbate 80, tyloxapol, polyoxyl stearates, polyethoxylated castor oils, poloxamers, polaxamines, medium and long chain fatty acids and phospholipids. The concentration of the surfactant in the formulation is about 0.01-3%, preferably 0.01-2%, more preferably 0.1-1% w/v. The tonicity agent is present in an amount to achieve a final formulation tonicity between 220-360 mOsm/kG, preferably 250-340 mOsm/kG, and most preferably between 260 and 320. The tonicity agent can be non-ionic or ionic. Non-ionic tonicity agents include diols, such as glycerol, mannitol, erythritol; and sugars such as dextrose. Other non-ionic tonicity agents such as polyethylene glycol, propylene glycol, which also function as cosolvents, can also be used. The non-ionic tonicity agent is in general in an amount of 0-20%, preferably 0-10%, more preferably 0-5%. Preferred non-ionic agents are glycerol, mannitol and dextrose, in an amount 2-6%.

The tonicity agent can also be ionic agents such as sodium chloride, potassium chloride, a balanced salt solution, sodium phosphate, or sodium citrate. The ionic tonicity agents can be present in an amount of 0.3-1.5%, preferably 0.6-0.9%.

The surfactants, the tonicity agent, the buffer and any other ingredients introduced into the formulation must have good solubility in water, and have compatibility with other components in the formulation. Health regulations in various countries require that multi-dose ophthalmic preparations shall include a preservative. Many well known preservatives that have been used in some other ophthalmic preparations cannot be used in the present invention, since those preservatives are not considered safe for repeated ocular use, or they interact with the surfactant employed herein to form a complex that reduces the bactericidal activity of the preservative. In one embodiment, benzalkonium chloride is employed as a safe preservative; benzalkonium chloride may be used with disodium edetate (EDTA), a chelating agent, to enhance its antimicrobial activity. Other suitable preservatives included benzyl alcohol, methyl parabens, propyl parabens, chlorobutanol, borate and benzethonium chlorides. Typically, such preservatives are employed at a level of from 0.001-1%, preferably, 0.001-0.25%, and most preferably 0.001-0.2%.

Optionally, the formulation can include a viscosity enhancer to increase the resident time of the formulation on the ocular surface. The viscosity enhancer must not cause ocular discomfort. Hydroxypropyl methyl cellulose, for example, is an acceptable viscosity enhancer for the present invention.

In one embodiment, the pharmaceutical formulation comprises 0.5-0.9% ionic tonicity modifier such as sodium chloride; the formulation contains additional buffering agents (such as sodium phosphates) at 5-100 mM, a surfactant within a range of 0.01-3%, a preservative in a range of 0.001-0.1%, a chelating agent in a range of 0.01-0.5% w/v, and pH adjusters. Such an aqueous composition has a tonicity of 220-360 mOsm/kG and is formulated at pH 6.3-7.8.

In another embodiment, the pharmaceutical formulation comprises 1-3% non-ionic tonicity agent such as glycerol; the formulation contains buffering agents (such as sodium phosphates and/or sodium citrate and citric acid) within a range of 5-50 mM, a surfactant within a range of 0.01-2%, a chelating agent in a range of 0.005-0.5% w/v, and pH adjusters. Such an aqueous composition has a tonicity of 220-360 mOsm/kG and is formulated at pH 6.3-7.8. The formulation optionally contains a preservative in a range of 0.001-0.1% w/v.

The present invention is also directed to an aqueous pharmaceutical formulation comprising 0.001-2% ROCK inhibitor compound, 0.02-0.25% polaxamer, and a tonicity agent to maintain a tonicity between 220-360 mOsm/kG. The formulation optionally comprises 1-100 mM buffer to maintain the pH between 6.3-7.8. Suitable buffers include phosphate, citrate, maleate, or a combination thereof. Phosphate buffer is preferred.

In one embodiment, the pharmaceutical formulation of the present invention is administered topically to the eye in the form of ophthalmic drops. The pharmaceutical formulations of the present invention are made by aseptic technique or are terminally sterilized. The solutions of the invention are prepared by thoroughly mixing the ROCK inhibitor compound, buffer, tonicity modifier, surfactant, chelating agent; optionally, non-ionic polymers, complexing agents, solubilizing agents, preservatives and antioxidant agent.

A discovery towards this invention relates to the aqueous solubility and formulation stability in relation to pH. Inventors have unexpectedly found that a more soluble and stable formulation is at the lower pH range of acceptable topical ophaltimc formulations (pH 4.5), rather than the higher pH range. The stability and solubility of the formulation decrease with increasing pH. However, at pH 6.3-7.8, the stability and solubility of the pharmaceutical formulation of the present invention is acceptable. Thus, the current formulation has improved therapeutic properties with acceptable aqueous solubility and long term stability. At pH higher than 7.8, the stability of the compounds is not acceptable and the ocular tolerability decreases.

The pharmaceutical formulation can be sterilized by filtering the formulation through a sterilizing grade filter, preferably of a 0.22 micron nominal pore size. The pharmaceutical formulation can also be sterilized by terminal sterilization using one or more sterilization techniques, including but not limited to a thermal process, such as an autoclaving process.

The pharmaceutical formulations of the present invention are useful as agents for modulation of wound healing after trabeculectomy. The pharmaceutical formulations in general are less toxic to corneal endothelial cells than the antimetabolites such as 5-fluorouracil or mitomycin C. The pharmaceutical formulations inhibit actomyosin-driven contractility, leading to deterioration of the actin microfilament system and perturbation of its membrane anchorage, which weakens the cell-extracellular matrix adhesions. These properties inhibit wound healing and thereby reduce bleb failure following the surgery.

The pharmaceutical formulation of the present invention is useful as agents for lowering intraocular pressure, and is thus useful in the treatment or prevention of glaucoma or associated ophthalmic conditions.

The pharmaceutical formulation of the present invention is useful in the treatment or prevention of neurodegenerative diseases as a consequence of increased intraocular pressure and damage to the ocular neurons.

The present invention provides a method of reducing intraocular pressure, a method of treating glaucoma, and a method of inhibiting wound healing after trabeculectomy. The method comprises the step of administering to a subject in need of treatment the pharmaceutical formulation of the present invention, in an amount effective to alter the actin cytoskeleton, such as by inhibiting actin polymerization.

The pharmaceutical formulation disclosed herein can be administered to the eyes of a patient by any suitable means, but are preferably administered in the form of drops, spray or gel-forming aqueous solution. Alternatively, the pharmaceutical formulation can be applied to the eye via aqueous formulations of liposomes, micelles, emulsions, and/or microemulsions. Further, the pharmaceutical formulation can be infused onto the tear film via a pump catheter system. In another embodiment, the pharmaceutical formulation is contained within a continuous or selective release device, for example, membranes such as, but not limited to, those employed in the Ocusert® System (Alza Corp., Palo Alto, Calif.) or Retisert (Bausch & Lomb, Rochester, N.Y.). As an additional embodiment, the pharmaceutical formulation can be contained within, carried by, or attached to contact lenses that are placed on the eye. Another embodiment of the present invention involves the pharmaceutical formulation contained within a swab or sponge that can be applied to the ocular surface. Another embodiment of the present invention involves the pharmaceutical formulation contained within a liquid spray that can be applied to the ocular surface. Another embodiment of the present invention involves an injection of the pharmaceutical formulation directly into the lacrimal tissues or onto the eye surface.

The invention is illustrated further by the following examples that are not to be construed as limiting the invention in scope to the specific procedures described in them. It is believed that one skilled in the art can, using the preceding description, utilize the present invention to its fullest extent. The following preferred specific embodiments therefore are to be construed as merely illustrative, and not limiting the disclosure in any way whatsoever.

The invention is illustrated further by the following examples that are not to be construed as limiting the invention in scope to the specific procedures described in them.

### EXAMPLES

### Example 1

### Impact of pH on Solubility of Compounds

Solubility was determined within a target pH range of 4.0-9.0 using a buffered cosolvent system (plON pSOL Evolution). The results in Table 1 show that Compound 2.039 (A) had solubility >20 mg/mL at pH 4-5.8, Compound 1.123 (B) had maximum solubility of 5 mg/mL at pH 5.6. These results indicate that as the pH increases, the solubility of the compounds decreases.

TABLE 1
Impact of pH on Solubility of Compound A and B.

| | | Avg. Sol |
|---|---|---|
| Compound | pH | (μg/mL) |
| A | 4.0 | >20000 |
| | 5.8 | >20000 |
| | 7.7 | 2312 |
| B | 5.6 | 5001 |
| | 7.2 | 948 |
| | 8.5 | 282 |

Another study was performed in which Compound A was prepared in a vehicle which contained a tonicity agent (NaCl) and a non-ionic surfactant, to determine the solubility of the compound at a target pH of 7.3. Concentrations of the surfactant used were at the maximum allowable concentration for excipients Generally Regarded As Safe (GRAS). A 20 mM concentration of the compound was prepared in 0.85% NaCl, then filtered and analyzed by UV to determine the concentration of Compound A. The results show that formulations containing polysorbate 80 and poloxamer 407 gave the highest solubility of Compound A. Therefore, polysorbate 80 and poloxamer 407 can counteract some of the loss of solubility at neutral pH about 7.3.

TABLE 2
List of Surfactants and Resulting Compound A Concentrations

| Excipient | Concentration | | Concentration |
|---|---|---|---|
| (surfactant or | of | pH of | (mg/mL) |
| chelating agent) | Surfactant | Solution | Compound A |
| EDTA | 0.3% | 7.39 | 1.38 |
| Poloxamer 407 | 0.16% | 7.28 | 6.02 |
| Polysorbate 80 | 1% | 7.49 | 3.85 |
| Poloxamer 188 | 0.1% | 7.30 | 1.49 |
| HCO-40 | 0.5% | 7.39 | 2.54 |
| (hydrogenated | | | |
| castor oil) | | | |

### Example 2

### Stability of Compound A (Accelerated)

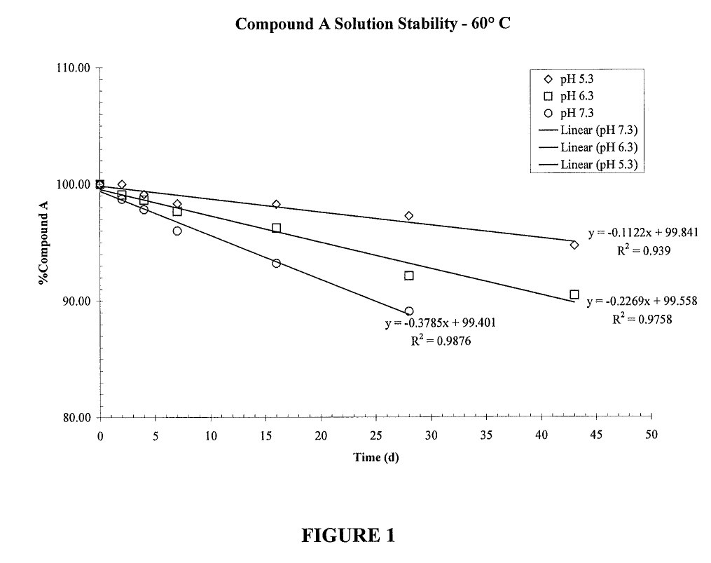

Compound A was formulated in a 0.9% saline solution containing 0.1% EDTA, 0.01% Benzalkonium Chloride and 0.8% Polysorbate 80 at three levels of pH; 5.3, 6.3 and 7.3. In order to determine the effects of pH on the stability of the compound, the solutions were stored at 60° C. and analyzed by HPLC using UV detection FIG. 1 shows that an increase in pH from pH 5.3 to pH 6.3 and 7.3 caused a decrease in stability of Compound A due to chemical degradation.

### Example 3

### Effect of pH on Ocular Surface, Aqueous Humor and Systemic Bioavailability

Dose Formulation and Administration. Compound A was formulated at 0.12% w/v (the equivalent millimolar concentration is 3 mM) in 10 mM phosphate, 0.8% polysorbate 80, 0.85% NaCl, 0.01% BAC, 0.1% EDTA at three different pH's, 5.3, 6.3 and 7.3, Compound A was administered as a 30 μl drop to both eyes of each animal within a dosing group and the influence of pH on ocular and systemic exposure was examined.

Study sampling. Plasma, aqueous humor, and ocular samples were obtained from 2 animals (4 eyes) per dosing group at times of 0.25, 0.5, 1, and 2 hours post dosing.

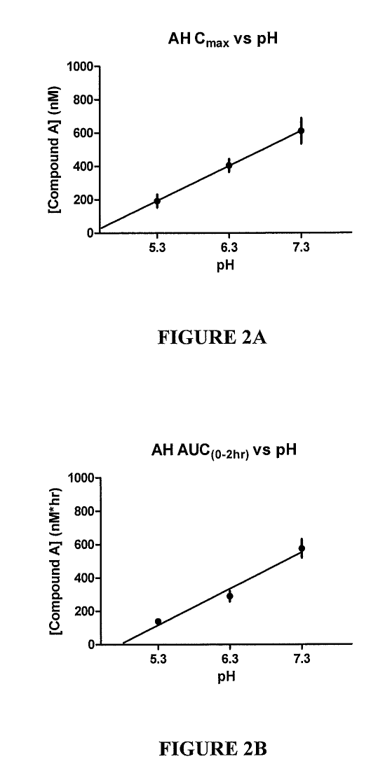

FIG. 2A shows the aqueous humor Cmax vs. pH. FIG. 2B shows the aqueous humor AUC vs. pH. Aqueous humor is the fluid within the anterior chamber of the eye and has the closest correlation to the concentration at the site of action, the trabecular meshwork. Cmax indicates the peak concentration of drug found within aqueous humor. AUC indicates total concentration of the drug within the aqueous humor over time. The results of FIGS. 2A and 2B indicate that increasing pH in the formulation enhances the exposure within the aqueous humor.

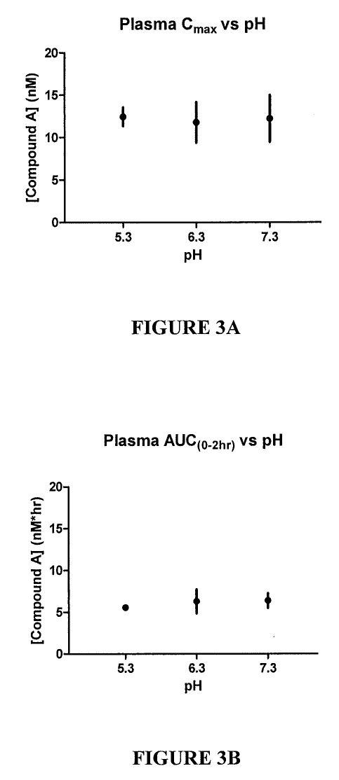

FIG. 3A shows the plasma humor Cmax vs. pH. FIG. 3B shows the plasma AUC vs. pH. Plasma concentration analysis indicates systemic exposure. Systemic exposure is the exposure of the drug to the entire body. Cmax and AUC describe the peak concentration and total concentration of the drug over time, respectively, for Compound A in plasma. The results of FIGS. 3A and 3B indicate that increasing pH of the formulation has no effect on the plasma concentration of the compounds.

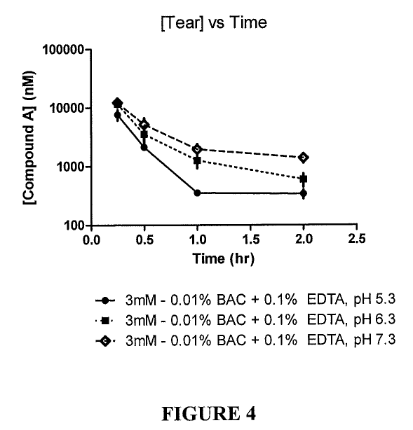

FIG. 4 shows the ocular surface concentration of Compound A over time. 40 μL of saline was applied to the eyes at 0.25 h, 0.5 h, 1 h, and 2 h after administration of Compound A, and the lavage fluids were collected as samples. Ocular surface relates to the surface of the cornea and conjunctiva. Ocular surface residence time is the average time Compound A resides on the ocular surface. FIG. 4 shows an increase in residence time when the pH of the Compound A formulation was increased from 5.3, 6.3 to 7.3. At 0.25 h, the ocular concentration of Compound A increased only slightly between pH 5.3 and 6.3, with no change from 6.3 to 7.3. The later time points had a greater separation in the ocular concentration of Compound A between pH 5.3-7.3. The ocular concentration of Compound A remained consistent between 1 and 2 hours for pH 7.3. This data indicates higher pH increased residence time over the 2 hour period, thus allowing more drugs to penetrate into the anterior chamber.

These studies demonstrate that when pH of the formulation increases, ROCK-inhibiting compounds exhibit an increase in residence time on the ocular surface and exhibit an increase in concentration within the aqueous humor of the anterior chamber, while exhibit no effect on systemic exposure.

### Example 4

### Effect of Concentration and Formulation on Ocular Comfort

Dose Formulation and Administration. Compound 2.039 was formulated at 0.16% w/v in four different formulations A-D as follows.

Formulation A: 10 mM phosphate, 1% polysorbate 80, 0.85% NaCl, 0.02% BAC, 0.2% EDTA pH 7.0.

Formulation B: 10 mM phosphate, 1% polysorbate 80, 2.36% Glycerol, 0.02% BAC, 0.2% EDTA pH 7.1.

Formulation C: 10 mM phosphate, 1% polysorbate 80, 2.36% Glycerol, 0.02% BAC, 0.2% EDTA, 0.5% hydroxypropyl methyl cellulose, pH 7.1.

Formulation D: 10 mM phosphate, 1% polysorbate 80, 2.36% Glycerol, 0.02% BAC, 0.2% EDTA, 1% 1,2 dimyristoyl-sn-glycero-3-phosphocholine, pH 7.1.

Pharmacodynamic Analysis. Formulations A-D were administered as two 30 μl drops to the right eye of each rabbit within a dosing group. The rabbits were evaluated for 15 minutes after ocular instillation and their changes in behavior were recorded. A composite score for each rabbit within each treatment group was created based upon the number of times they demonstrated a unilateral blink, bilateral blink, front pay wipe of the face, scratch and head shake. The higher the score, the more discomfort an animal is. A mean±SE was generated for each group.

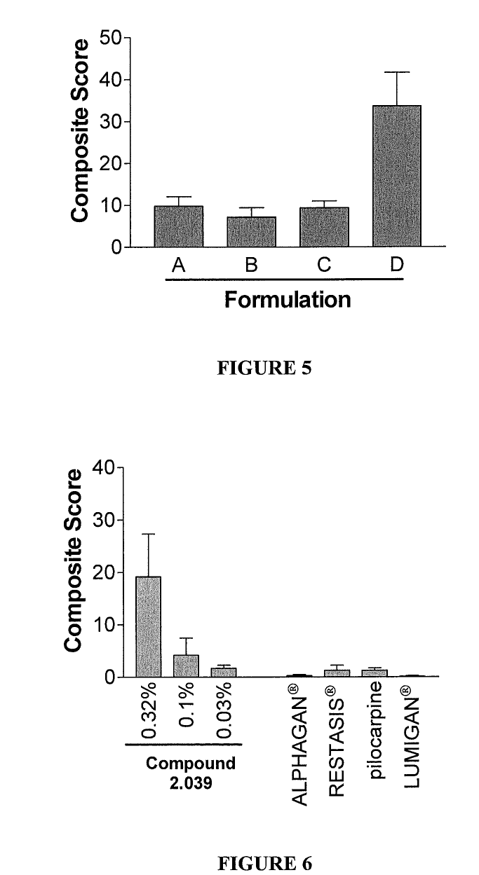

FIG. 5 shows ocular comfort scores vs. formulations. FIG. 5 shows that the addition of certain adjuvant such as 1,2 dimyristoyl-sn-glycero-3-phosphocholine, which is a viscosity enhancer and increased ocular surface residence, increased ocular discomfort.

Compound 2.039 was then formulated at 0.03%, 0.1% and 0.32% w/v in formulation A above and the effect of concentration on ocular surface comfort was examined in comparison to other approved glaucoma medications. The results are shown in FIG. 6.

FIG. 6 shows ocular comfort scores vs. concentrations of Compound A. FIG. 6 indicates that increasing concentrations of a ROCK-inhibiting compound increased ocular discomfort. However, the ocular comfort at the lower concentrations (0.03 and 0.1% w/v) is comparable to those of the approved glaucoma medications such as ALPHAGAN®, RESTASIS®, pilocarpine, and LUMIGAN®.

What is claimed:

1. An aqueous pharmaceutical formulation comprising at least one ROCK inhibitor having Formula II in an amount of 0.01-0.4% w/v, a non-ionic surfactant in an amount of 0.01-2% w/v, and a tonicity agent to maintain a tonicity between 220-360 mOsm/kG, at a pH between 6.3 to 7.8, wherein the ROCK inhibitor, the surfactant, and the tonicity agent are compatible in the formulation; 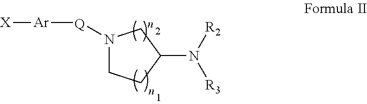
wherein:
Q is C═O, SO₂, or (CR₄R₅)ₙ₃;
n₁ is 1, 2, or 3;
n₂ is 1 or 2;
n₃ is 0, 1, 2, or 3;
wherein the ring represented by 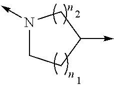
is optionally substituted by alkyl, halo, oxo, OR₆, NR₆R₇, or SR₆;
R₂ is R₂-1 or R₂-2, optionally substituted: 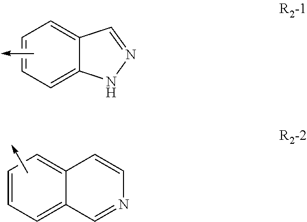
Ar is a monocyclic or bicyclic aryl or heteroaryl ring;
X is from 1 to 3 substituents on Ar, and each is independently selected from the group consisting of OR₈, NR₈R₉, SR₈, SOR₈, SO₂R₈, SO₂NR₈R₉, NR₈SO₂R₉, CONR₈R₉, NR₈C(═O)R₅, NR₈C(═O)OR₉, OC(═O)NR₈R₉, and NR₈C(═O)NR₉R₁₀, R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl heteroarylalkyl, heteroarylalkenyl, heteroarylalkynyl, (heterocycle)alkyl, (heterocycle)alkenyl, (heterocycle)alkynyl or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, and NR₁₄C(═O)NR₁₅R₁₆;
wherein any two of the groups R₈, R₉ and R₁₀ are optionally joined with a link selected from the group consisting of bond, —O—, —S—, —SO—, —SO₂—, and —NR₁₇— to form a ring;
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle;
with the first proviso that if X is acyclic and is connected to Ar by a carbon atom, then X contains at least one nitrogen or sulfur atom,
with the second proviso that if X is acyclic and is connected to Ar by an oxygen or nitrogen atom, then X contains at least one additional oxygen, nitrogen or sulfur atom, and
with the third proviso that if X is connected to Ar by a —SO₂— linkage, then R₂ is not nitrogen- or oxygen-substituted R₂-2.

2. An aqueous pharmaceutical formulation comprising at least one ROCK inhibitor having Formula II in an amount of 0.01-0.4% w/v, a non-ionic surfactant in an amount of 0.01-2% w/v, and a tonicity agent to maintain a tonicity between 220-360 mOsm/kG, at a pH between 6.3 to 7.8, wherein the ROCK inhibitor, the surfactant, and the tonicity agent are compatible in the formulation; 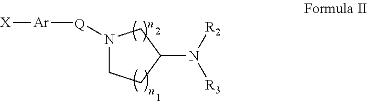
wherein:
Q is C═O, SO₂, or (CR₄R₅)ₙ₃;
n₁ is 1, 2, or 3;
n₂ is 1 or 2;
n₃ is 0, 1, 2, or 3;
wherein the ring represented by 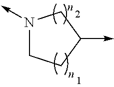
is optionally substituted by alkyl, halo, oxo, OR₆, NR₆R₇, or SR₆;
R₂ is R₂-1 or R₂-2, optionally substituted: 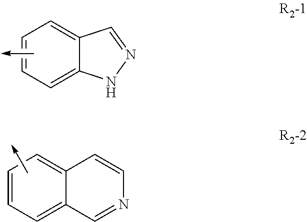
Ar is a monocyclic or bicyclic aryl or heteroaryl ring;
X is from 1 to 3 substituents on Ar, each independently in the form Y-Z, in which Z is attached to Ar;
Y is one or more substituents on Z, and each is independently selected from the group consisting of H, halogen, OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, and NR₈C(═O)NR₉R₁₀;
Z is alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, and (heterocycle)alkynyl;
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl or heterocycle; optionally substituted by one or more halogen or heteroatom-containing substituents selected from the group consisting of OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅ NR₁₄C(═O)R₁₅, NR₁₄C(═O)OR₁₅, OC(═O)NR₁₄R₁₅, and NR₁₄C(═O)NR₁₅R₁₆;
wherein any two of the groups R₈, R₉ and R₁₀ are optionally joined with a link selected from the group consisting of bond, —O—, —S—, —SO—, —SO₂—, and —NR₁₇— to form a ring; and
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl or heterocycle.

3. An aqueous pharmaceutical formulation comprising at least one ROCK inhibitor having Formula II in an amount of 0.01-0.4% w/v, a non-ionic surfactant in an amount of 0.01-2% w/v, and a tonicity agent to maintain a tonicity between 220-360 mOsm/kG, at a pH between 6.3 to 7.8, wherein the ROCK inhibitor, the surfactant, and the tonicity agent are compatible in the formulation; 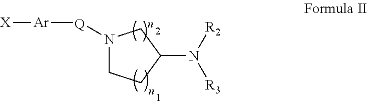
wherein:
Q is C═O, SO₂, or (CR₄R₅)ₙ₃;
n1 is 1, 2, or 3;
n₂ is 1 or 2;
n₃ is 0, 1, 2, or 3;
wherein the ring represented by 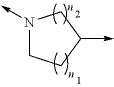
is optionally substituted by alkyl, halo, oxo, OR₆, NR₆R₇, or SR₆;
R₂ is R₂-1 or R₂-2, optionally substituted: 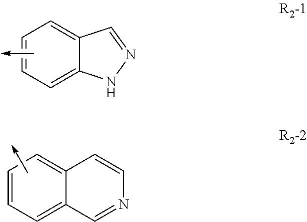
Ar is a monocyclic or bicyclic aryl or heteroaryl ring;
X is from 1 to 3 substituents on Ar, each independently in the form Y-Z, in which Z is attached to Ar;
Y is one or more substituents on Z, and each is independently OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀,
Z is alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl;
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁ SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, or NR₁₄C(═O)NR₁₅R₁₆;
wherein any two of the groups R₈, R₉ and R₁₀ are optionally joined with a link selected from the group consisting of bond, —O—, —S—, —SO—, —SO₂—, and —NR₁₇— to form a ring; and
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle;
with the proviso that when Z is selected from the group consisting of alkyl, alkenyl, and alkynyl, and Y falls on the carbon by which Z is attached to Ar, then Y contains at least one nitrogen or sulfur atom.

4. An aqueous pharmaceutical formulation comprising at least one ROCK inhibitor having Formula II in an amount of 0.01-0.4% w/v, a non-ionic surfactant in an amount of 0.01-2% w/v, and a tonicity agent to maintain a tonicity between 220-360 mOsm/kG, at a pH between 6.3 to 7.8, wherein the ROCK inhibitor, the surfactant, and the tonicity agent are compatible in the formulation; 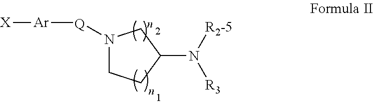
wherein:
Q is C═O, SO₂, or (CR₄R₅)ₙ₃;
n₁ is 1, 2, or 3;
n₂ is 1 or 2;
n₃ is 0, 1, 2, or 3;
wherein the ring represented by 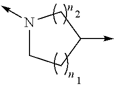
is optionally substituted by alkyl, halo, oxo, OR₆, NR₆R₇, or SR₆; R₂-5 is 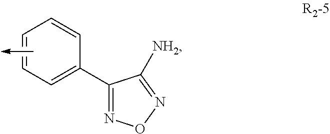
optionally substituted;
Ar is a monocyclic or bicyclic aryl or heteroaryl ring;
X is from 1 to 3 substituents on Ar, each independently in the form Y-Z, in which Z is attached to Ar;
Y is one or more substituents on Z, and each is independently selected from the group consisting of H, halogen, OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, and NR₈C(═O)NR₉R₁₀;
Z is independently selected from the group consisting of absent, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, heterocycle, (heterocycle)alkyl, (heterocycle)alkenyl, and (heterocycle)alkynyl;
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl cycloalkenyl, cycloalkylalkyl, cycloalkylalkenyl, cycloalkylalkynyl, heteroaryl heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₉ and R₁₀ are independently H, alkyl, alkenyl, alkynyl, aryl, arylalkyl, arylalkenyl, arylalkynyl, cycloalkynyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl or heterocycle; optionally substituted by one or more halogen or heteroatom-containing substituents selected from the group consisting of OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, and NR₁₄C(═O)NR₁₅R₁₆;
wherein any two of the groups R₈, R₉ and R₁₀ are optionally joined with a link selected from the group consisting of bond, —O—, —S—, —SO—, —SO₂—, and —NR₁₇— to form a ring; and
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl or heterocycle.

5. The aqueous pharmaceutical formulation according to claim 1, wherein said non-ionic surfactant is selected from the group consisting of: polysorbates, tyloxapol, polyoxyl castor oil, polaxamers, polyethylene glycol, caprylic triglyceride, polyoxyl stearates, glyceryl monostearate, and combination thereof.

6. The aqueous pharmaceutical formulation according to claim 5, wherein said non-ionic surfactant is a polysorbate, a polaxamer, or a combination thereof.

7. The aqueous pharmaceutical formulation according to claim 1, further comprising 1-100 mM buffer suitable to maintain the pH between 6.3-7.8.

8. The aqueous pharmaceutical formulation according to claim 7, wherein said buffer is citrate buffer, phosphate buffer, maleate buffer, or combination thereof.

9. The aqueous pharmaceutical formulation according to claim 1, further comprising a chelating agent and/or a preservative.

10. The aqueous pharmaceutical formulation according to claim 1, wherein said tonicity agent is a non-ionic tonicity agent.

11. The aqueous pharmaceutical formulation according to claim 10, wherein said non-ionic tonicity agent is glycerol, mannitol or dextrose.

12. The aqueous pharmaceutical formulation according to claim 1, wherein said tonicity agent is an ionic tonicity agent.

13. The aqueous pharmaceutical formulation according to claim 1, wherein said ROCK inhibitor is (R)-2-(3-((3-(isoquinolin-5-ylamino)pyrrolidin-1-yl)methyl)phenoxy)ethanol.

14. The aqueous pharmaceutical formulation according to claim 1, wherein said ROCK inhibitor is (R)—N-(3-((3-(1H-indazol-5-ylamino)piperidin-1-yl)methyl)phenyl)ethane-sulfonamide.

15. The aqueous pharmaceutical formulation according to claim 1, wherein said ROCK inhibitor is in an amount of 0.03-0.2% (w/v).

16. The aqueous pharmaceutical formulation according to claim 1, wherein said pH is 6.3-7.3.

17. A method for reducing intraocular pressure in a subject in need thereof, comprising the steps of:
identifying a subject in need thereof, and
administering to the subject the aqueous pharmaceutical formulation according to claim 1, in an amount effective to inhibit actomyosin interactions.

18. The method according to claim 17, wherein said method treats glaucoma.
